(19) United States
(12) Patent Application Publication
(10) Pub. No.: US 2003/0119057 A1
(43) Pub. Date: Jun. 26, 2003

(54) Forming and modifying dielectrically-engineered microparticles

(75) Inventors: Peter R.C. Gascoyne, Bellaire, TX (US); Jody Vykoukal, Houston, TX (US); Daynene Vykoukal, Houston, TX (US); Susan Sharma, Sugarland, TX (US); Frederick F. Becker, Houston, TX (US)

Correspondence Address: Michael C. Barrett, FULBRIGHT & JAWORSKI L.L.P., 600 CONGRESS AVENUE, SUITE 2400, AUSTIN, TX, 78701, US

(73) Assignee: Board of Regents

(21) Appl. No.: 10/027,782

(22) Filed: Dec. 20, 2001

(51) Int. Cl. C12Q 1/68; G01N 33/53; C12N 5/08; C08B 37/00

(52) U.S. Cl. 435/7.1; 435/7.5; 435/372; 536/53

(57) ABSTRACT

Engineered microparticles, libraries of microparticles, and methods relating thereto. The microparticles are distinguishable based on differences in dielectric response to an applied electric field. In different embodiments, the dielectric differences may be engineered through, but not limited to, dielectrically dispersive materials, surface charge, and/or fluorescence. Gangliosides may be incorporated with the microparticles to control aggregation. Vesicles including erythrocyte ghosts may be used as a basis for microparticles. The microparticles may utilize a biotin streptavidin system for surface functionalization.

Methodology of the current disclosure may be used with the apparatuses and methods described in U.S. Pat. No. 6,294,063, which is expressly incorporated herein by reference.

Other patents and applications that may be used in conjunction with the current disclosure include U.S. Pat. No. 5,858,192, entitled “Method and apparatus for manipulation using spiral electrodes,” filed Oct. 18, 1996 and issued Jan. 12, 1999; U.S. Pat. No. 5,888,370 entitled “Method and apparatus for fractionation using generalized dielectrophoresis and field flow fractionation,” filed Feb. 23, 1996 and issued Mar. 30, 1999; U.S. Pat. No. 5,993,630 entitled “Method and apparatus for fractionation using conventional dielectrophoresis and field flow fractionation,” filed Jan. 31, 1996 and issued Nov. 30, 1999; U.S. Pat. No. 5,993,632 entitled “Method and apparatus for fractionation using generalized dielectrophoresis and field flow fractionation,” filed Feb. 1, 1999 and issued Nov. 30, 1999; U.S. patent application Ser. No. 09/395,890 entitled “Method and apparatus for fractionation using generalized dielectrophoresis and field flow fractionation,” filed Sep. 14, 1999; U.S. patent application Ser. No. 09/883,109 entitled “Apparatus and method for fluid injection,” filed Jun. 14, 2001; U.S. patent application Ser. No. 09/882,805 entitled “Method and apparatus for combined magnetophoretic and dielectrophoretic manipulation of analyte mixtures,” filed Jun. 14, 2001; U.S. patent application Ser. No. 09/883,112 entitled “Dielectrically-engineered microparticles,” filed Jun. 14, 2001; and U.S. patent application Ser. No. 09/883,110 entitled “Systems and methods for cell subpopulation analysis,” filed Jun. 14, 2001, each of which are herein expressly incorporated by reference.

Yet another application that may be used in conjunction with the teachings of the current invention include those described in “Micromachined impedance spectroscopy flow cytometer of cell analysis and particle sizing,” Lab on a Chip, vol. 1, pp. 76-82 (2001), which is incorporated by reference.

The government may own rights in aspects of the present invention pursuant to contract number N66001-97-C-8608 from SPAWAR under the Defense Advanced Research Project Agency Order No. E934. The government may also own rights in aspects of the present invention pursuant to grant no. DAAD19-00-1-0515 from the Army Research Office and grants 1R21 CA88364-01 and 1R33 CA88364-01 from the National Cancer Institute.

### FIELD OF THE INVENTION

The present invention relates generally to the fields of chemistry and the life-sciences. More particularly, it concerns techniques for the manipulation, separation, purification, and indexing of target analytes using dielectrically-engineered microparticles (which may be referred to herein as DEMPs).

### DESCRIPTION OF RELATED ART

Improved methods for separating and identifying chemicals, cells and biomolecules have been fundamental to many advances in chemistry and the life sciences. Much of the discovery process is based on determining qualitative and quantitative information about a particular chemical, cell, biomolecule or other analyte. Analysis methods such as filtration, centrifugation, spectroscopy and light microscopy typically exploit differences in the inherent physical properties of analytes to achieve analyte separation and/or detection.

Improvements in these basic methods have generally fallen into two categories: (i) the resolving ability, or sensitivity, of the method has been improved to better differentiate subtle physical differences between analytes, or (ii) a substance, or label, with certain properties that are readily discernable has been coupled to an analyte to make the analyte detectable or easier to resolve indirectly. Gradient centrifugation and high performance liquid chromatography are examples of methods based on increasing the sensitivity of an existing method; cell-staining with fluorescent antibodies and biomolecule radiolabeling are examples of improved methods based on coupling labels to analytes to facilitate resolution of an analyte. Although these improvements have exhibited degrees of success in the field, problems remain. Notably, these methods still do not allow for a method whereby analytes may be indexed, detected, and manipulated at once, nor do they allow for the separate manipulation of many different types of analytes at once.

One type of traditional analysis that makes use of labels is termed a “one-pot” reaction. One-pot reactions are those where reagents are simply added to a sample aliquot in a single test tube or beaker. Any molecules of target analyte present in the sample react with the added indicator to yield a calorimetric, fluorescent or chemiluminescent product or complex. This reaction product or complex is then detected and, usually, quantified. The defining feature of such methods is that the detectable species exists only when the target analyte is actually present in the sample.

One example of a useful label for a one pot analysis is a molecular beacon. Molecular beacons utilize a molecule that has a built in fluorophore and a quencher. The fluorophore and quencher are held in close proximity until such time that molecule is bound to a target. At that time, they are pulled sufficiently farther apart so that the fluorophore can fluoresce. When the quencher no longer quenches, the target can be observed via the fluorescence.

Other examples of one-pot assays include calorimetric pH detection or non-specific labeling of nucleic acids using an intercalating dye, such as ethidium bromide. Techniques such as southern and northern blotting for nucleic acids and ELISA and western blotting for proteins use labeled probes that bind to and facilitate the detection of specific biochemical analytes. These antibody or nucleic acid probes are radioactive, fluorescent, or enzymatically active whether or not they are bound to their target analyte:

In order for the above methods to yield useful results, however, it is necessary to distinguish between the unbound analyte, the free probe, and the analyte-probe complexes. This is accomplished by immobilizing the analyte-probe complexes to a solid support and then washing away the free, unbound molecules, leaving only the labeled analyte-probe complexes attached to the solid support. Unfortunately, this process may be somewhat complicated and time-consuming.

In order to solve at least some of the problems inherent in traditional identification and separation of analytes, certain microparticles have been used. In the late 1970's, techniques were developed that enable relatively straightforward production of uniformly sized microparticles in the submicron to 100-micron size range. Later, techniques for making microparticles having magnetic properties were also developed. Microparticles produced using these techniques have been linked to various probes that interact with or bind to specific target analytes or classes of analytes to form microparticle-analyte complexes. In this way, microparticles can be made to act as labels that are specific for target analytes.

Existing microparticle labels may be divided broadly into two categories, namely those for analyte detection and those for analyte manipulation. In both categories, labels sensitized against a specific target analyte are added to a sample and incubated under conditions that facilitate binding of the target analyte, if present in the sample, to the microparticle-based label to form a microparticle-analyte complex.

In existing analyte detection protocols, certain physical properties of the microparticle, such as fluorescence, opacity to light or other radiation, or emission of radiation have been exploited as reporters to infer the presence of the microparcle-analyte complex. For example, a metallic microparticle or nanoparticle that complexes with a target protein in a cell via an antibody probe can be observed and quantified by electron microscopy and used to infer that the target protein analyte is in specific locations in the cell and is an example of a label used in detection protocols.

In detection protocols, it is not the analyte that is detected directly. Instead, the presence of the analyte is inferred by its association with the microparticle reporter, an association mediated by the interaction of the probe and the analyte. Detection protocols can also include two-step labeling methods in which a secondary label is used to reveal the presence of the microparticle-analyte complex. In this case, the analyte attaches to a first probe on the microparticle and then the analyte is subsequently labeled with a second specific label which is then used as the reporter that allows the presence of the analyte to be inferred. Because the analyte ends up being effectively sandwiched between the microparticle and a second, reporter label, such a double labeling protocols are familiarly termed “sandwich assays” in the art.

In manipulation protocols, microparticle-based labels are used as “handles” to assist in the physical manipulation of analytes. In such protocols, certain physical properties of the microparticle such as density, electrical charge, or size are exploited to isolate, separate or otherwise manipulate the microparticle-analyte complex. In such methods the analyte is not manipulated directly. Instead, the microparticle (i.e., “the handle”) is manipulated and any analyte bound to the microparticle, is manipulated indirectly based on its association with the microparticle. Manipulation protocols based on microparticle labels unfortunately require additional analysis steps to identify the target analyte.

Although the above microparticle-based systems have exhibited at least a degree of utility in this field, the necessary additional steps of identifying a target (apart from manipulating the target) represent extra time and cost to the scientist or engineer. Further, even with the use of microparticles, it is sometimes the case that the detection of the microparticle itself does not necessarily infer the presence of the target analyte. Still further, traditional microparticles do not allow for the simultaneous, separate manipulation of many different types of analytes. Simply put, traditional microparticles do not allow for the indexing of different analytes followed by simultaneous manipulation, detection, and/or identification. In other words, traditional techniques do not allow for the creation of a library of different probes that may each bind to different targets and allow for simultaneous manipulation, identification, and detection of the different species.

Certain problems, weaknesses, or shortcomings mentioned above are not meant to be exhaustive; other problems are know to exist within the art. However, the above discussion demonstrates that a need exists for improved methodology relating to manipulation, separation, purification, and indexing of analytes.

### SUMMARY OF THE INVENTION

Engineered microparticle labels made and used according to the present disclosure can be designed to overcome limitations discussed above because analyte indexing may be achieved. In particular, the present disclosure allows for the simultaneous identification, manipulation, and detection of a variety of different target analytes through the use of a library of DEMPs having different, distinguishable dielectric properties. Alternatively, the present disclosure overcomes limitations in the art because it allows analyte binding to be detected. Specifically, microparticle labels with dielectric properties that are sensitive to analyte binding can be used to confirm analyte binding by sensing the change in the AC electrokinetic behavior of the label following the binding.

Uses for engineered microparticles according to embodiments disclosed herein are vast and include, but are not limited to, blood analysis; disease detection and characterization; clinical preparation of pure cell populations; the detection and identification of pathogens in food processing, public water distribution systems, agriculture, and the environment; the separation of subcellular compartments, the purification of stem cells for bone marrow transplants, and the purging or collection of diseased cells for both diagnostic and research purposes. In addition, engineered microparticle may be applied to molecular analyses including the isolation, separation, purification and identification of various materials such as proteins and nucleic acids. Further, techniques disclosed herein may be used in conjunction with current methods of separating cells, such as flow cell cytometry.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a dielectrically-engineered microparticle according to one embodiment of the present disclosure.

FIG. 2A and FIG. 2B are dielectrophoretic force diagrams. The diagrams show the dielectrophoretic force vectors experienced by a spherical particle of radius 5 μm in a rotating field produced by phase quadrature voltage signals of 1 Vᵣₘₛ applied to the electrodes. FIG. 2A. Re(f_{CM})=0.5 and Im(f_{CM})=0. In FIG. 2A, the force directs particles towards the electrode located along the edges of the figures. In FIG. 2B Re(f_{CM})=0 and Im(f_{CM})=0.5. The forces in FIG. 2B direct particle circular translation about the center of the electrode geometry.

FIG. 3 shows a graph of electrorotation spectra. In particular, typical ROT spectra for erythrocytes (Δ), T-lymphocytes (◯) and MDA231 breast cancer cells (□) in isotonic sucrose of conductivity 56 mS/m are shown. Curves show best fits of a single-shell dielectric model, discussed below.

FIG. 4 shows a graph of AC electrokinetic behavior of different microparticle types. The cDEP (conventional Dielectrophoresis) and twDEP (travelling wave DEP) response for five different microparticle types are shown. Each microparticle type is identical except for the thickness of their outermost shells, which vary from about 1-10 nm. Each different type of microparticle may be linked to a different probe and used to label and then manipulate or identify different analytes in a sample mixture.

FIG. 5 is a schematic diagram showing the separation of analytes. Three different types of engineered microparticles according to one embodiment of the present disclosure (denoted a, b and c) with AC electrokinetic properties such as those illustrated in FIG. 4, are sensitized with antibodies for CD3, CD4 and CD18 cell surface antigens to form labels for different cell subpopulations. These labels facilitate DEP-FFF (DEP/field flow fractionation) separation of CD3⁺, CD4⁺ and CD18⁺ cells as shown in the simulated DEP-FFF fractogram.

FIG. 6 is a schematic diagram showing the detection of analyte binding. The dielectric properties of engineered microparticles may be perturbed by analyte binding. An AC electrokinetic analysis method such as DEP-FFF may be used to detect this change in the form of elution peak broadening or elution peak shifting.

FIG. 7 is a graph showing the dependence of particle velocity on dielectric properties.

FIG. 8 is a schematic illustrating particle and medium polarization.

FIGS. 9A-15B show dielectrically-engineered microparticles, their properties, and behavior according to one embodiment of the present disclosure.

FIG. 16 is a schematic illustrating sandwich (double label) assays that may be used for detecting protein and mRNA in studies in accordance with the present disclosure.

FIG. 17 shows a dielectrically-engineered microparticle according to one embodiment of the present disclosure including a polystyrene core, a gold shell, and an alkanethiol self-assembled monolayer.

FIG. 18 shows a dielectrically-engineered microparticle according to one embodiment of the present disclosure including a polystyrene core, a gold shell, an alkanethiol self-assembled monolayer, and a phospholipid self-assembled monolayer.

FIG. 19 shows a dielectrically-engineered microparticle according to one embodiment of the present disclosure including a polystyrene core, a gold shell, an alkanethiol self-assembled monolayer, and a cross-linked phospholipid self-assembled monolayer.

FIG. 20 shows a dielectrically-engineered microparticle according to one embodiment of the present disclosure including a polystyrene core, a gold shell, an alkanethiol self-assembled monolayer, a phospholipid self-assembled monolayer, and a nucleic acid probe.

FIG. 21 shows a dielectrically-engineered microparticle according to one embodiment of the present disclosure including a polystyrene core, a gold shell, an alkanethiol self-assembled monolayer, a phospholipid self-assembled monolayer, and a protein probe.

FIG. 22 is a graph illustrating crossover frequency versus conductivity.

FIG. 23 is a graph showing data including dispersive, frequency range data. The solid lines show dielectric loss that gives rise to traveling wave dielectrophoresis and electrorotation while the dashed lines show dielectric permittivity (dielectric constant) that gives rise to conventional dielectrophoresis.

FIG. 24 is a graph illustrating the frequency responses of two types of microparticles: three engineered through a self-assembled, insulator-over-conductive-core, biomimetic approach, and three through a dielectric-dispersive-core approach. The biomimetic microparticles exhibit a low permittivity at low frequencies that increases with increasing frequency. The dispersive-core microparticles exhibit a high permittivity at low frequencies that decreases with increasing frequency.

FIG. 25 is a schematic diagram of a microparticle incorporating gangliosides for affecting aggregation according to embodiments of the present disclosure.

FIG. 26 is a schematic diagram showing chemical details of an embodiment of the present disclosure utilizing gangliosides for aggregation control.

FIG. 27 is a graph, including instructive commentary, that illustrates dielectrophoretic spectral response to changes in vesicle properties according to embodiments of the present disclosure.

FIG. 28 is a graph showing dielectrophoresis data for resealed erythrocyte ghosts according to embodiments of the present disclosure.

FIGS. 29A-29C are schematic diagrams showing constituent microparticles within an exemplary microparticle library according embodiments of the present disclosure.

FIG. 30 is a graph illustrating dielectric properties of the microparticles of FIGS. 29A-29C.

FIGS. 31A-31C generally illustrate a biotin/streptavidin system for surface functionalization according to embodiments of the present disclosure.

FIG. 32 is a schematic diagram showing addressable, indexible microparticles for multiplex analyte detection and manipulation according to embodiments of the present disclosure.

FIG. 33 is a chemical schematic diagram showing particular embodiments concerning surface functionalization using biotinylated phospholipids.

### DESCRIPTION OF ILLUSTRATIVE EMBODIMENTS

This disclosure describes a new technology in which microparticles are designed and produced with certain predetermined, or engineered, dielectric and/or magnetic properties such that their AC electrokinetic (including conventional dielectrophoresis (cDEP), traveling wave dielectrophoresis (twDEP), traveling wave dielectrophoresis (gDEP) or electrorotation (ROT)) and magnetophoretic (MAP) behavior is at least partially calculable or controllable.

These engineered-microparticles may be sensitized with various probes such as antibodies, nucleic acids or chemical ligands by methods known in the art and correspondingly used to label a variety of different analyte types including, but not limited to, cells, subcellular components, and biomolecules. Analytes labeled with such engineered microparticles may then be manipulated using existing AC electrokinetic or magnetophoretic methods (or a combination thereof).

Since different classes of engineered microparticles may be designed with different AC electrokinetic and/or magnetophoretic responses, several different analytes in a mixture can simultaneously be labeled with one or more probes and then individually (or as a group defined by each type of different probe) addressed, manipulated and/or identified. This ability may be referred to as indexing and represents a significant advance over existing technology. Further, as new AC electrokinetic and magnetophoretic analysis methods are developed, the engineered microparticle labels discussed herein may be used with those methods to address, manipulate, and identify analytes.

In addition, previously disclosed AC electrokinetic analysis methods such as dielectrophoretic field-flow fractionation (DEP-FFF), travelling-wave DEP (twDEP), and spiral electrode methods may be enhanced through the use of the engineered microparticle labels discussed herein. These previously disclosed methods typically exploit differences in the inherent dielectric properties of analytes to achieve analyte manipulation and identification. Probe-sensitized engineered-microparticles, on the other hand, provide techniques for separating analytes with unknown or indiscriminable dielectric properties and for identifying analytes by sensing changes in engineered microparticle behavior following analyte binding. Engineered dielectric and magnetic microparticle labels are therefore an enabling technology that may provide powerful new methods for separating and identifying analytes in many diverse fields.

Several types of engineered microparticles may be used to simultaneously label and probe a sample for multiple target analytes. Because each microparticle type may be engineered to have a specific, distinguishable dielectric and/or magnetic response, different target analytes in the mixture may be independently manipulated, sequentially or in parallel.

Additionally, engineered microparticles according to the present disclosure, may be sensed, and hence identified, by methodology known in the art.

Additionally, engineered microparticles may be discriminated, sorted and processed by AC electrokinetic and/or magnetophoretic methods. Because the discrimination of some AC electrokinetic based methods is orders of magnitude better than existing isolation methods, is controllable by electronic means, and unlike existing methods, is applicable to integrated and automated Microsystems for chemical and biological analysis, the engineered microparticles of this disclosure may be used to solve many, if not all, of the shortcomings addressed above in relation to the existing technology in this field.

Different engineered microparticle types, each designed with unique intrinsic dielectric properties, may be indexed based upon their dielectric properties and used as frequency dependent dielectric handles to manipulate different analytes simultaneously. A library of engineered microparticles may then be developed with different dielectric properties. Such a library may be used to perform indexing. The library may also be used to develop bead-based biochemical assays for several different types of applications such as for microflumes.

In one embodiment, analytes may be detected using a sandwich protocol. In such a system, a change in bead fluorescence is the result of two separate binding events, mediated by the presence of a specific analyte. Engineered microparticles may first be sensitized (or linked) with a capture probe that has high binding affinity for a specific analyte. These sensitized engineered microparticles may then incubated with a sample droplet, resulting in the formation of engineered microparticle-analyte complexes. A droplet containing a labeling probe with high affinity for a secondary epitope or nucleic acid sequence on the analyte may then added to the reaction mixture, resulting in the formation of engineered microparticle-analyte-flurophore complexes. These complexes may be pulled to a reaction surface using positive dielectrophoresis and held in situ while the suspending droplet is pulled away. A different reagent droplet may then be moved over the engineered microparticle complexes and the DEP force removed. The engineered microparticles may be spontaneously released into and thermokinetically mixed with the new reagent, resulting in a buffer change or washing operation. This ability to reversibly immobilize analytes in a microfluidic device without the use of a probe that is permanently linked to the surface of the device represents a major advance in microflume-based chemical analysis.

Calibration, sample carryover, and cross-contamination problems known in the art may be addressed by using molecular recognition and sensing elements that are attached to engineered microparticles so that a new aliquot of sensitized engineered microparticles can be used for each and every assay. By disposing of the microparticles afterwards, by running a “blank” between each sample, and by allowing for cleaning cycles, calibration issues may be addressed and the absence of carryover and cross-contamination can be verified. Placing biologically active components on engineered microparticles also means that a single fluidic device may be applied to a wide range of sample preparation and molecular analysis problems by using different engineered microparticle/probe combinations. Finally, because no biological components need to be attached to fixed surfaces those surfaces may be PTFE coated, for example, to reduce biomolecular adhesion and carryover issues. It follows that the decision to use the engineered microparticles of the present disclosure may enhance the potential applicability of the technology by allowing a single device to have multiple applications.

Fabricating engineered microparticles according to the present disclosure creates the opportunity to conduct molecular analyses in parallel using a cocktail of different engineered microparticle/probe combinations. Assays using engineered beads require minimal quantities of sample. For example, a engineered microparticle of 5 μm diameter has the relatively large surface area of approximately 78 μm² yet occupies a volume of only 65 fL, about 1/15 that of a typical tumor cell. 100 tumor cells and 250 engineered microparticles comprised of 10 different engineered microparticle types may be packed into spherical region of 50 μm diameter using DEP-mediated focusing. This is the equivalent of almost 10⁹ cells/ml held in contact with 2×10⁹ engineered microparticles/ml carrying the molecular probes. The time for hybridization of target mRNA's to cDNA probes on magnetic microparticle surfaces has been shown to be just a few minutes in concentrated cell lysates. Therefore, the engineered microparticle-based approach described herein, using such high cell and engineered microparticle concentrations, has the potential to enable rapid assays for molecular markers in an integrated system.

In developing engineered microparticle-based indexing technology of the present disclosure, a reduced panel of 10 or so key molecular markers may be selected from a library of available markers for the purpose of screening for specific subsets of suspected disease states. By combining sample preparation and molecular analysis into a single, automated process, this system allows for the exploitation of gene-chip-derived molecular epidemiological data and render it accessible to a wide population.

Engineered Microparticles

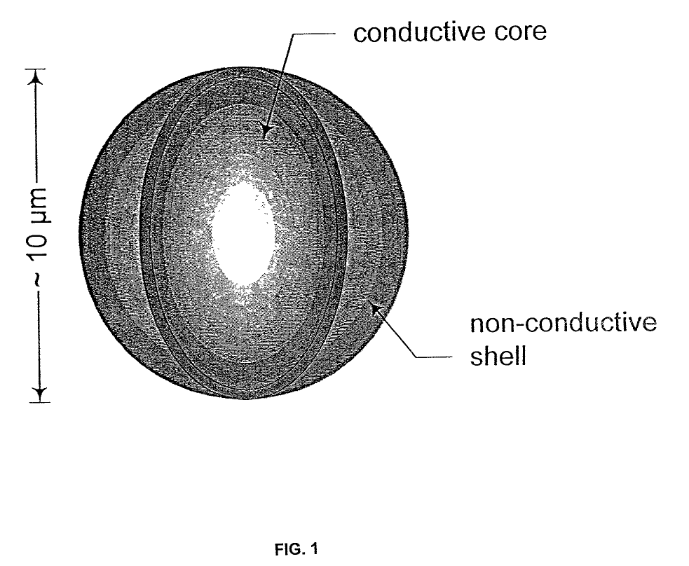

The structure of an engineered microparticle according to one embodiment of the present disclosure is shown in FIG. 1. There, a conductive core surrounded by a thin, poorly conducting, dielectric shell is illustrated. The conductive core may be made of a wide variety of materials. Further, the conducting core may be solid or hollow. Still further, the conducting core may be formed from an insulating inner region surrounded in whole, or in part, by a conducting outer region.

Although the shape of the inner core may vary somewhat, the shape may be spherical in one embodiment. In other embodiments, however, the shape may be elliptical or any other suitable shape.

The dielectric shell may be formed from a number of materials suitable to create desired dielectric properties, and specifically, properties that will provide for the dielectrophoretic responses at given frequencies. The dielectric shell may be coupled to the inner conductive core by any manner known in the art.

The shape of the outer dielectric shell may vary as well, but in one embodiment, it may generally be spherical or, generally, conform to the shape of the inner conductive core.

The size, composition, thickness, and shape of the conductive core and/or the dielectric shell may all be adjusted and optimized so as to achieve desired dielectric and/or magnetic properties. In particular, the sizes, thicknesses and compositions may be adjusted so that an engineered microparticle has the proper dielectric properties to be manipulated by a certain range of dielectrophoretic forces.

In one embodiment, polystyrene-coated silver microparticles may be used as engineered microparticles. These engineered microparticles undergo a frequency-dependent change from a non-conducting state to a conducting state. This is the result of a dielectric dispersion in which an AC field of appropriate frequency penetrates through the non-conducting polystyrene shell.

In another embodiment, a fabrication process using self-assembled monolayers (SAMs) of alkanethiolate on gold or silver-coated, hollow glass (or polystyrene or other microparticle) microparticles may be used to produce improved biomimetic particles. The dielectrophoretic behavior of these engineered microparticles may be predicted using established dielectrophoretic and multi-shell models known in the art, and the effects of changing engineered microparticle properties such as particle diameter and insulating layer thickness and composition may be determined by methods known in the art.

The engineered microparticle of FIG. 1 may be designed to mimic a mammalian cell. Specifically, it may be engineered so that its AC electrokinetic behavior mimics that of mammalian cells. This behavior has been characterized extensively for cells and is distinguished by a well-defined and relatively sharp frequency dependence known in the art. Samples of these microparticles have been produced by encapsulating conductive core particles of silver with non-conducting shells of various thicknesses of polystyrene. The cDEP response of these particles has been studied and shown to vary in accordance with the predictions of established AC electrokinetic theory.

Classes of engineered microparticles different than that illustrated in FIG. I may be designed and produced according to manufacturing principles known in the art. Again, within each structural class of microparticles, a range of different dielectric responses may be achieved by varying the compositions, thicknesses, and/or other properties of the layers comprising the individual microparticles. In this way, a library of engineered microparticles having well defined, yet clearly distinguishable, dielectric and/or magnetic properties may be produced. By designing and fabricating different microparticle types with distinct dielectric and/or magnetic properties, each type of engineered microparticle may be independently addressed, manipulated, and characterized even when it is part of a mixture of multiple types of engineered microparticles.

Linking Elements

According to one embodiment, the engineered microparticles discussed above may be coupled to one or more linking elements, or probes, in order to act as engineered microparticle labels. The general use of different linking elements is known in the art. However, sections below explain several specific embodiments relating to different linking elements that may be used in conjunction with the engineered microparticles described above. Those having skill in the art, having the benefit of the present disclosure, will recognize that other linking elements may, however, be used.

The term “linking element” or “probe” as used herein refers to any component that has an affinity for another component termed here as a “target analyte.” The binding of the linking element to the target analyte forms an affinity pair between the two components.

For example, such affinity pairs include, for instance, biotin with avidin/streptavidin, antigens or haptens with antibodies, heavy metal derivatives with thiogroups, various polynucleotides such as homopolynucleotides as poly dG with poly dC, poly dA with poly dT and poly dA with poly U. Any component pairs with strong affinity for each other can be used as the affinity pair. Suitable affinity pairs are also found among ligands and conjugates used in immunological methods.

The choice of linking element will obviously depend on the nature of the microparticle and the target analyte. For instance, if one wishes to capture a nucleic acid species (the target analyte) on a microparticle, the linking element will normally be chosen to be a nucleic acid or nucleic acid analogue oligomer having a sequence complementary to that of the target analyte or a part thereof.

The linking element may be bound first to the microparticle and may then be a species having an affinity for the target analyte. Preferably, the affinity for the target analyte is a selective affinity such that the formation of the complex between the microparticle and the target analyte is selective and provides at least a degree of identification of the target analyte. Preferably, the affinity is highly specific and accordingly the linking element bound to the particle, which provides the selective affinity for the target analyte, may be an antibody or an antibody fragment having antibody activity, an antigen, a nucleic acid probe or a nucleic acid analogue probe having selective affinity for complementary nucleic acid sequences, or avidin or an avidin-like molecule such as strept-avidin.

Nucleic Acids as Linking Elements

Nucleic acid based linking elements may be synthetic oligonucleotides, single-stranded DNA, complimentary DNA (cDNA), and RNA. Although shorter oligonucleotides may be easier to make, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 300, 350, 400, 450, 500, 550, 600, 650, 700, 750, 800, 850, 900, 950, 1000, 1100, 1150, 1200, 1250, 1300, 1500, 1922, 2000, 3000, 4000 bases and longer are contemplated as well.

Antibodies as Linking Elements

Antibody based linking elements refers to monoclonal or polyclonal antibodies, single chain antibodies, or recombinantly engineered antibodies. As used herein, the term “antibody” is intended to refer broadly to any immunologic binding agent such as IgG, IgM, IgA, IgD and IgE. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Generally, IgG and/or IgM are preferred because they are the most common antibodies in the physiological situation and because they are most easily made in a laboratory setting. Means for preparing and characterizing antibodies are well known in the art (See, e.g., Antibodies: A Laboratory Manual, Cold Spring Harbor Laboratory, 1988, incorporated herein by reference).

Methods for generating polyclonal antibodies are well known in the art. Briefly, a polyclonal antibody is prepared by immunizing an animal with an antigenic composition and collecting antisera from that immunized animal. A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal can be bled and the serum isolated and stored, and/or in some cases the animal can be used to generate monoclonal antibodies (MAbs). For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or the desired antibody fraction may be purified by well-known methods, such as affinity chromatography using another antibody or a peptide bound to a solid matrix.

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner that effectively stimulates antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and have enzyme deficiencies that render them incapable of growing in certain selective media that support the growth of only the desired fused cells (hybridomas).

Any one of a number of myeloma cells may be used, as are known to those of skill in the art (Goding, 1986). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate.

Fusion procedures usually produce viable hybrids at low frequencies, about 1×10⁶ to 1×10⁻⁸. However, this low frequency does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and thus they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such as HPLC or affinity chromatography.

Large amounts of the monoclonal antibodies may also be obtained by multiplying hybridoma cells in vivo. Cell clones are injected into mammals that are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present disclosure, fragments of the monoclonal antibody may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention can be synthesized using an automated peptide synthesizer, or by expression of full-length gene or of gene fragments in E. coli.

Other Linking Elements

Other linking elements include peptides, antitumor agents, antibiotics and other therapeutic compounds. Again, what is required of these linking elements is the ability to bind with a degree of specificity to a target analyte. The use of peptides, antitumor agents, antibiotics and other therapeutic compounds would allow the ability to identify or purify such target analytes as receptors, cofactors, enzymes, or any other target capable of binding to the linking element.

Examples of the peptide linking elements include LH-RH antagonists (see U.S. Pat. Nos. 4,086,219, 4,124,577, 4,253,997 and 4,317,815), insulin, somatostatin, somatostatin deruvatives (see U.S. Pat. Nos. 4,087,390, 4,093,574, 4,100,117 and 4,253,998), growth hormone, prolactin, adrenocorticotropic hormone (ACTH), melanocyte-stimulating hormone (MSH), thyrotropin-releasing hormone (TRH), their salts and derivatives (see JP-A 50-121273 and JP-A 52-116465), thyroid-stimulating hormone (TSH), luteinizing hormone (LH), follicle-stimulation hormone (FSH), vasopressin, vasopressin derivatives, oxytocin, calcitonin, parothyroid hormone, glucagon, gastrin, secretin, pancreozymin, cholecystokinin, angiotensin, human placental lactogen, human chorionic gonadotropin (HCG), enkephalin, enkephalin derivatives (see U.S. Pat. No. 4,277,394 and EP-A 31,567); and polypeptides such as endorphin, kyotorphin, interferon (α-type, β-type, γ-type), interleukin (I to XI), tuftsin, thymopoietin, tymosin, thymosthymlin, thymic hormone factor (THF), serum thymic factor (FTS) and derivatives thereof (see U.S. Pat. No. 4,299,438), tumor necrosis factor TNF), colony stimulating factor (CSF), motilin, deinorphin, bombesin, neurotensin, caerulein, bradykinin, urokinase, asparaginase, kallikrein, substance P, nerve growth factor, blood coagulation factor VIII and IX, lysozyme chloride, polymyxin B, colistin, gramicidin, bacitracin, protein synthesis-stimulating peptide (see G. B. Patent No. 8,232,082), gastric inhibitory polypeptide (GIP), vasoactive intestinal polypeptide (VIP), platelet-derived growth factor (PDGH), growth hormone-releasing factor (GRF, somatoclinine), bone morphagenetic protein (BMP), epidermal growth hormone (EGF) and the like.

Examples of an antitumor agent include bleomycin hydrochloride, methotrexate, actinomycin D, mitomycin C, vinblastine sulfate, vincristine sulfate, daunorubicin hydrochloride, adriamynin, neocarzinostatin, cytosine arabinoside, fluorouracil, tetrahydrofuryl-5-fluorouracil, picibanil, lentinan, levamisole, bestatin, azimexon, glycyrrhizin, poly A:U, poly ICLC and the like.

Examples of an antibiotic include gentamicin, dibekacin, kanendomycin, lividomycin, tobromycin, amikacin, fradiomycin, sisomysin, tetracycline, oxytetracycline, roliteracycline, doxycycline, ampicillin, piperacillin, ticarcillin, cefalotin, cefaloridine, cefotiam, cefsulodin, cefmenoxime, cefmetazole, cefazollin, cefataxim, cefoperazone, ceftizoxime, moxolactame, thienamycin, sulfazecine, azusleonam, salts thereof, and the like.

Examples of therapeutic drugs include antipyretic, analgesic and anti- inflammatory agents such as salicylic acid, sulpyrine, flufenamic acid, diclofenace, indometacin, morphine, pethidine, levorphanol tertrate, oxymorphone and the like; antitussive expectorants such as ephedrine, methylephedrine, noscapine, codeine, dihydrocodeine, alloclamide, chlorphezianol, picoperidamine, cloperastine, protokylol, isoproterenol, salbutamol, terebutaline, salts thereof and the like; sedatives such as chlorpromazine, prochloperazine, trifluoperazine, atropine, scopolamine, salts thereof and the like; muscle relaxant such as pridinol, tubocurarine, pancuronium and the like; antiepileptic agents such as phenytoin, ethosuximide, acetazolamide, chlordiazepoxide and the like; antiulcer agents such as metoclopramide, histidine and the like; antidepressant such as imipramine, clomipramine, onxiptiline, phenelzine and the like; antiallergic agent such as diphenhydramine hydrochloride, chlorpheniramine malate, tripelennamine hydrochloride, methdilazine hydrochloride, clemizole hydrochloride, diphenylpyraline hydrochloride, methoxyphenemine hydrochloride and the like; cardiotonics such as transpieoxocamphor, terephylol, aminophylline, etilifrine and the like; antianrythmic agents such as propranolol, alprenolol, bufetololoxyprenolol and the like; vasodilators such as oxyfedrine, diltiazem, tolazoline, hexobendine, bamethan and the like; hypotensive diuretics such as hexamethonium bromide, pentolinium, mecamylamine, ecarazine, clonidine and the like; antidiabetic agents such as glymidine, glipizide, phenformin, buformin, metformin and the like; anticoagulants such as heparin, citric acid and the like; hemostatic agents such as thromboplastin, thrombin, menadione, acetomenaphthone, ε-aminocaproic acid, tranexamic acid, carbazochrome sulfonate, adrenochrome monoaminoguanidine and the like; antituberculous agents such as isoniazid, ethambutol, para-aminosalicylic acid and the like; hormones agents such as prednisolone, dexamethasone, betametasone, hexoestrol, methymazole and the like; narcotic antagonists such as levallorphan, nalorphine, naloxone, salts thereof and the like.

Attaching Linking Elements to Engineered Microparticles

The nature of the method used to convert an original engineered microparticle into a recognition element by complexing it with a linking element can vary widely according to the nature of the linking element.

In some cases, the complex may involve a crosslinking agent connecting the engineered microparticle and the linking element. The complex may further include a label connected to the linking element or microparticle, optionally Via a second linking element. The complex may involve numerous linking elements bound to the particle.

Antibodies and antibody fragments having antibody properties may be used for attachment. There are techniques suitable for coating antibodies on to the surface of microparticles which are well known to those skilled in the art. Antibody coated particles are capable of recognizing and binding corresponding antigens which may be presented on micro-organism cells or some other target analyte.

Methods are also known for binding oligo-nucleic acid probes to microparticles, such as the engineered microparticles described herein. Suitable techniques are by way of example described in Patent Application No. WO 93/04199. Where the linking element is a nucleic acid probe or a nucleic acid analogue probe, the resulting microparticle will of course be suitable for recognizing and binding complementary nucleic acid sequences.

Other methods of attaching linking elements to microparticles involve the use of functionalized crosslinking agents. Such crosslinking reagents are well known to those of skill in the art. A useful reference describing the scope of crosslinkers that are commonly available and their uses and limitations may be found in the Pierce Chemical Catalogue (Rockford, Ill.).

Labels

The use of an additional label to further increase the detectability of an engineered microparticle as well as to alter its magnetic and electrokinetic characteristics may be utilized. For instance, antibodies bearing fluorophores or chromaphores may be bound to an engineered microparticle so that the complex so-formed can be further distinguished from the starting engineered microparticle by magnetic and/or electrokinetic means as well as detection by fluorescence or color.

Such a label may be bound to the microparticle or linking element either before, simultaneously with, or after the formation of the complex between the target analyte and the engineered microparticle. The label may include a second linking element carried by the label. Once again, it is preferred that the affinity for the target analyte possessed by the second linking element is selective, preferably highly specific and the second linking element may also be an antibody, an antibody fragment having antibody activity, an antigen, a nucleic acid probe, a nucleic acid analogue probe, avidin or an avidin-like molecule. The use of a label of this nature may be desired to aid ready detection of the complex and/or where a complex between the microparticle and the target analyte does not in itself possess sufficiently distinctive magnetic and electrokinetic properties, thus the magnetic and electrokinetic may be further altered by the inclusion in the complex of the label. To this end, the label may be a fluorophore or chromaphore, or a micro- organism, a metal particle, a polymer bead or a magnetic particle. A suitable material is colloidal gold which is easily bound to antibodies (as the second species) to form a label. Antibodies bound to colloidal gold are commercially available and methods for binding antibodies to colloidal gold are for instance described in Geohegan et al (1978). Other metal particles however may be employed, e.g. silver particles and iron particles.

The use of a label of the kind described above may be suitable even where a complex between the ligand and a particle possesses sufficiently distinctive magnetic and electrokinetic properties to enable the formation of such a complex to be observed. A higher level of specificity may in certain cases be obtained by the use of a label in such a complex. Thus for instance, one may wish to distinguish a micro-organism expressing an antigen A from a micro-organism expressing antigens A and B. This may be accomplished by the use of engineered microparticles having as a linking element an antibody to A and a label having as it's linking element an antibody to B. The difference in the characteristics of the labeled complex (between the engineered microparticle, the microorganism and the label) and the unlabeled complex (between the engineered microparticle and the micro-organism) can be observed, and used to distinguish microorganisms expressing antigen A only, from those expressing A and B.

Labels for both cells and smaller particles can include fluorescent markers, e.g. FITC or rhodamine, chromophores, luminescent markers or enzyme molecules which can generate a detectable signal. Examples of the latter include luciferases and alkaline phosphatase. These markers may be detected using spectroscopic techniques well known to those skilled in the art.

Exemplary Microparticle-based Labels and Their Uses

The following engineered microparticle-based labels are included to further delineate uses of the present disclosure. In as much as the following patent applications and publications describe linking elements, crosslinkers or methods of bonding or attaching linking elements to microparticles, target analytes, and others methodologies that may be employed in the present invention, they are herein incorporated by reference.

A method for determining the concentration of substances in biological fluids (e.g., drugs, hormones, vitamins and enzymes) wherein magnetically responsive, permeable, solid, water insoluble, microparticles are employed is disclosed in U.S. Pat. No. 4,115,534.

U.S. Pat. No. 4,285,819 describes microparticles which may be employed to remove dissolved ions from waste aqueous streams by formation of chelates. U.S. Pat. No. 3,933,997 describes a solid phase radio immunoassay for digoxin where anti-digoxin antibodies are coupled to magnetically responsive particles.

Small magnetic particles coated with an antibody layer are used in U.S. Pat. No. 3,970,518 to provide a large and widely distributed surface area for sorting out and separating select organisms and cells from populations thereof. U.S. Pat. No. 4,018,886 discloses small magnetic particles used to provide a large and widely distributed surface area for separating a select protein from a solution to enable detection thereof. The particles are coated with a protein that will interact specifically with the select protein.

U.S. Pat. No. 4,070,246 describes compositions comprising stable, water insoluble coatings on substrates to which biologically active proteins can be covalently coupled so that the resulting product has the biological properties of the protein and the mechanical properties of the substrate, for example, magnetic properties of a metal support.

A diagnostic method employing a mixture of normally separable protein-coated particles is discussed in U.S. Pat. No. 4,115,535. Microparticles of acrolein homopolymers and copolymer(s) with hydrophilic comonomers such as methacrylic acid and/or hyroxyethylmethacrylate are discussed in U.S. Pat. No. 4,413,070. U.S. Pat. No. 4,452,774 discloses magnetic iron-dextran microparticles which can be covalently bonded to antibodies, enzymes and other biological molecules and used to label and separate cells and other biological particles and molecules by means of a magnetic field. Coated magnetizable microparticles, reversible suspensions thereof, and processes relating thereto are disclosed in U.S. Pat. No. 4,454,234. A method of separating cationic from anionic beads in mixed resin beds employing a ferromagnetic material intricately incorporated with each of the ionic beads is described in U.S. Pat. No. 4,523,996. A magnetic separation method utilizing a colloid of magnetic particles is discussed in U.S. Pat. No. 4,526,681. U.K. Patent Application GB No. 2,152,664A discloses magnetic assay reagents.

An electron-dense antibody conjugate made by the covalent bonding of an iron-dextran particle to an antibody molecule is reported by Dutton et al. (1979). Ithakissios et al. (1977) describes the use of protein containing magnetic microparticles in radioassays. The separation of cells labeled with immunospecific iron dextran microparticles using high gradient magnetic chromatography is disclosed by Milday et al. (1984). Molday et al. (1982) describe an immuno-specific ferromagnetic iron-dextran reagent for the labeling and magnetic separation of cells. An application of magnetic microparticles in labeling and separation of cells is also disclosed by Molday et al. (1977). A solid phase fluoroimmunoassay of human albumin and biological fluids is discussed by Margessi et al. (1978). Nye et al. (1976) disclose a solid phase magnetic particle radioimmunoassay. Magnetic fluids are described by Rosenweig (1983). Magnetic protein A microparticles and their use in a method for cell separation are disclosed by Widder et al. (1979).

U.S. Pat. No. 5,279,936 is a method directed to the separation of a component of interest from other components of a mixture by causing the binding of the component of interest to magnetic particles. In the embodiment of the invention which is a method to separate cells from a mixture containing other components, the method comprises layering a first liquid medium containing cells and other components with a second medium which is of a different density than and/or different viscosity than the first liquid medium. The cells are bound to paramagnetic particles. The layered first liquid medium and the second liquid medium are subjected to a magnetic field gradient to cause the cell particles to migrate into the second medium. The purpose of isolating the cells in the second liquid medium is then, by a further embodiment, to separate the cells from the second liquid medium.

U.S. Pat. No. 4,935,147 is a method that specifically targets the application of magnetic separation in the assay of organic and inorganic biochemical analytes, particularly those analytes of interest in the analysis of body fluids. The method of this invention provides a way of separating non-magnetic particles from a medium by virtue of the chemically controlled non-specific reversible binding of such particles to magnetic particles. Because of the small size of the magnetic particles, it also provides for a very rapid binding of a substance to be separated. By then aggregating the particles there is provided a much more rapid and complete magnetic separation than has been achieved by previous methods.

Other current practices in the field for cell separation utilize matrix materials of, for example, hollow fibers, flat sheet membrane, or packed-bed bead or particle materials with physically adsorbed or covalently attached chemicals or antibodies for selective cell separation. These devices are designed to allow continuous whole blood or blood component inflow and return. Since these devices operate at normal blood flow rates under conditions in which the concentration of desired cells can be very low compared with other cell types, the separation process is often not efficient. Moreover, with these systems it is difficult to collect the selected cells in a viable state.

The development of paramagnetic beads offered the prospect of magnetic separation of target cells. Various methods to produce magnetic and paramagnetic particles are disclosed in the following United States patents: U.S. Pat. No. 4,672,040; U.S. Pat. No. 5,091,206; U.S. Pat. No. 4,177,253; U.S. Pat. No. 4,454,234; U.S. Pat. No. 4,582,622; U.S. Pat. No. 4,452,773; U.S. Pat. No. 5,076,950; U.S. Pat. No. 4,554,088; and U.S. Pat. No. 4,695,392.

Various methods were devised to use magnetic particles for assays. See, for example, United States patents: U.S. Pat. No. 4,272,510; U.S. Pat. No. 4,777,145; U.S. Pat. No. 5,158,871; U.S. Pat. No. 4,628,037; U.S. Pat. No. 4,751,053; U.S. Pat. No. 4,988,618; U.S. Pat. No. 5,183,638; U.S. Pat. No. 4,018,886; and U.S. Pat. No. 4,141,687.

Attempts were made to use magnetic particles for separation of biological components, including cells. The following is a list of United States patents known to the Applicants and believed to be directed to magnetic separators and methods: U.S. Pat. No. 4,855,045; U.S. Pat. No. 4,664,796; U.S. Pat. No. 4,190,524; U.S. Pat. No. 4,738,773; U.S. Pat. No. 4,941,969; U.S. Pat. No. 5,053,344; U.S. Pat. No. 5,200,084; U.S. Pat. No. 4,375,407; U.S. Pat. No. 5,076,914; U.S. Pat. No. 4,595,494; U.S. Pat. No. 4,290,528; U.S. Pat. No. 4,921,597; U.S. Pat. No. 5,108,933; U.S. Pat. No. 4,219,411; U.S. Pat. No. 3,970,518; and U.S. Pat. No. 4,230,685.

A number of techniques have been developed recently using microparticle-based methods to meet the demands for rapid and accurate detection of agents, such as viruses, bacteria and fungi, and detection of normal and abnormal genes. Such techniques, which generally involve the amplification and detection (and subsequent measurement) of minute amounts of target nucleic acids (either DNA or RNA) in a test sample, include inter alia the polymerase chain reaction (PCR) (Saiki et al., 1985; 1988; PCR Technology, Henry A. Erlich, ed., Stockton Press, 1989; Patterson et al., 1993), ligase chain reaction (LCR) (Barany, 1991), strand displacement amplification (SDA) (Walker et al., 1992), Qβ replicase amplification (QβRA) (Wu et al., 1992; Lomeli et al., 1989) and self-sustained replication (3SR) (Guatelli et al., 1990).

Other applications for such techniques include detection and characterization of single gene genetic disorders in individuals and in populations (see, e.g., Landergren et al., 1988 which discloses a ligation technique for detecting single gene defects, including point mutations). Such techniques should be capable of clearly distinguishing single nucleotide differences (point mutations) that can result in disease (e.g., sickle cell anemia) as well as deleted or duplicated genetic sequences (e.g., thalassemia).

Handles

If different types of engineered microparticle are linked to different probes that are directed against specific analytes, different target analytes may be simultaneously labeled yet independently manipulated within an analyte mixture. Upon binding to its target analyte, a sensitized engineered microparticle label (an engineered microparticle coupled to one or more linking elements or additional labels) acts as a handle that may be used to pull the analyte from cell lysate, serum or other biological sample, for example. Numerous labeled analytes may simultaneously be manipulated in a switchable, frequency dependent manner.

In addition to acting as handles, probe-sensitized engineered microparticles may also be used for detection and, in one embodiment, simultaneously, or near-simultaneous detection of analytes. With the benefit of this disclosure, engineered microparticles may be designed such that the dielectric properties and, thus, the dielectrophoretic behavior are very sensitive to analyte binding. The presence of a target analyte in a sample may be detected by observing this change in AC electrokinetic behavior.

In light of the above, it is apparent to those skilled in the art that the engineered microparticles of the present disclosure, which may be used for AC electrokinetic manipulation of cells and biomolecules, provide enabling technology for the development of improved separation and detection methods for integrated and automated microsystems, where conventional methods such as centrifugation or immunodetection are difficult or impractical to implement. Devices utilizing these improved methods may be useful in a variety of diagnostic and research applications, as discussed earlier.

In one embodiment, the isolation, identification, etc. of suspect cells from mixed cell suspensions and the manipulation of mixtures of dielectrically indexed engineered microparticles may be achieved, all in an integrated device. Achieving these steps ultimately depends upon ways of moving matter with respect to the solution that suspends it, a problem to which dielectrophoresis is ideally suited. Although principles of dielectrophoresis are known in the art, sections below explain, and apply, certain of those principles to the engineered microparticles discussed herein.

AC Electrokinetic Phenomena

AC electrokinetic phenomena are a family of related effects in which alternating electric fields induce forces on particles. These forces depend upon the dielectric characteristics of particles and their surroundings. The best-known electrokinetic phenomenon is conventional dielectrophoresis (cDEP). The term dielectrophoresis (DEP) was first used by Pohl to describe the motion of polarizable particles towards the minimum of dielectric potential in a non-uniform electric field (Pohl, 1978; Sauer, 1985; Kaler and Jones, 1990; Holzel et al., 1991; Gascoyne et al., 1993). This phenomenon is exploited in cell fusion and electroporation devices (Abidor et al., 1994; Wu et al., 1994) in order to bring cells into close contact through pearl chain formation. More recently, other electrokinetic phenomena including electrorotation (ROT, particle rotation resulting from the torque exerted on the particle by a rotating electrical field) (Arnold and Zimmermann, 1982 and 1988; Fuhr et al, 1990; Hu et al, 1990; Gimsa et al., 1991; Holzel and Lamprecht, 1992; Huang et al., 1992; Sukhorukov et al., 1993; Wang et al., 1994c) and travelling-wave dielectrophoresis (twDEP, lateral motion of a particle caused by an electrical field sweeping through space) (Masuda et al., 1988; Hagedorn et al., 1992; Huang et al., 1993 Gascoyne et al, 1994a) have been investigated for their applicability in the noninvasive characterization and manipulation of cells and biomolecules.

AC electrokinetic phenomena result from the interaction between an electric field and polarizations induced in a particle by the field. The effect has been studied in detail by several groups, and its theory is fairly well established (Pething, 1979; Jones, 1995). It is important to note that while dielectrophoresis and the more familiar electrophoresis both describe electrokinetic phenomena, they are distinguished by several fundamental differences. In dielectrophoresis particle motion is determined by the magnitude, polarity, and phase of charges that are induced in a particle by an applied field. It is not necessary that the particle carry an intrinsic net charge to experience dielectrophoresis. Electrophoresis, however, requires that a particle carry an intrinsic net charge. It is the interaction between this intrinsic charge and an electric field that causes particle motion. Furthermore, electrophoresis typically utilizes homogeneous, direct current electric fields. Dielectrophoresis requires the use of inhomogeneous electric fields that can be either direct or alternating current. The AC electrokinetic phenomena, cDEP, twDEP, gDEP and ROT, have been considered very little for separation and analysis in chemistry and the life sciences, despite the fact that they are, by their very nature, far more versatile than the commonly used method of electrophoresis. Consider the following advantages of AC electrokinetic methods:

(1) The magnitude and sign of the charges induced in a particle depend strongly on particle dielectric properties. In the case of engineered microparticles, this includes particle coating and core material properties. Particles with a wide range of dielectric properties can be made by changing the thickness and composition of the coating as well as the composition of the core particle.

(2) The magnitude and sign of the charges induced in a particle are not fixed but depend critically on the frequency of the applied field and on properties of the medium the particle is suspended in. For this reason, the engineered microparticles may be individually addressed in a frequency dependent manner.

(3) AC electrokinetic phenomena embody not just one type of linear motion but a variety of kinetic effects in two dimensions that can be exploited not only to manipulate particles but also to characterize their dielectric properties.

(4) As discussed in (1) and (2), the AC electrokinetic response of a particle is highly sensitive to the dielectric properties of the particle. Engineered microparticles may be produced such that their dielectric properties are very sensitive to binding of analyte. Such engineered microparticles provide a means of discriminating unbound engineered microparticles from analyte-microparticle complexes. This type of engineered microparticle may be used for qualitative, and in some cases quantitative, identification of an analyte.

(5) The strong dependencies of cell motions in two dimensions on the field configuration, the field frequency and the suspending medium dielectric properties promise versatility of particle separation technologies targeted at a variety of different applications.

All of these phenomena may be used individually or simultaneously to exploit fully the dielectric properties of the particles by appropriate applied field configurations.

Generalized Dielectrophoresis Theory

The dielectrophoretic force acting on a particle due to an imposed electrical field vector \vec{E}(t) can be written quite generally in terms of the effective dipole moment vector \vec{m}(t) that the field induces in the particle (Huang et al., 1992 and 1993; Gascoyne et al., 1994b; Wang et al., 1994a) as

\vec{F}(t)=(\vec{m}(t)·∇)\vec{E}(t) (1)

In the frequency domain, the induced particle dipole moment is given by

\vec{m}(ω)=4πεₘr³f_{CM}\vec{E}(ω) (2)

where ω is the angular frequency of the applied field, r the particle radius, and f_{CM} the Clausius-Mossotti factor defined as

$${f}_{CM} ( {ϵ}_{p}^{*} , {ϵ}_{m}^{*} ) = \frac{{ϵ}_{p}^{*} - {ϵ}_{m}^{*}}{{ϵ}_{p}^{*} + 2 {ϵ}_{m}^{*}} . \qquad ( 3 )$$

Here εₚ^{*} and εₘ^{*} are the complex permittivities of the particle and its suspending medium, respectively. Until recently, expressions describing different electrokinetic phenomena including cDEP and twDEP (Huang et al., 1993) were derived by substituting appropriate spatial expressions for \vec{E}, resulting in special cases of the force expression. However, by utilizing the fact that the mixed partial derivatives of the field with respect to space and time must obey the Swartz relationships (Gellert et al., 1977) in order that the field remain continuous, it has recently been derived (Wang et al., 1994a and 1994b) that the time-averaged dielectrophoretic force, may be represented as

<\vec{F}(t))=2πεₘr³(Re(f_{CM})∇E(rms)²+Im(f_{CM})(Eₓ₀²∇φₓ+E_{y0}²∇φ_{y}+E_{z0}²∇φ_{z})), (4)

where E(rms) is the rms value of the electric field strength. Eₜ₀ and φᵢ(i=x; y; z) are the magnitude and phase, respectively, of the field components in the principal axis directions. Unlike previous analyses, this expression can be used to investigate the forces arising from any form of applied field. It contains two terms that allow an appreciation, for the first time, that there are two independent force contributions to gDEP motion:

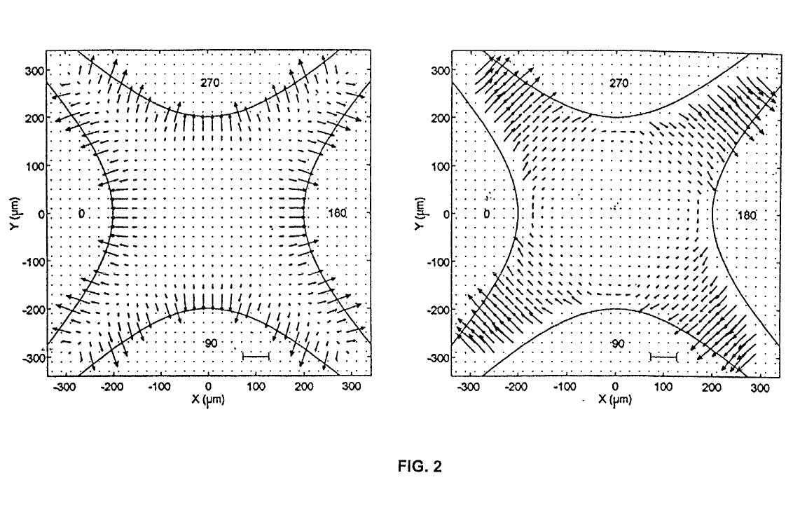

(i) the left hand term relates to the real (in-phase) component (Re(f_{CM})) of the induced dipole moment in the particle and to the spatial nonuniformity, ∇E(rms)², of the field magnitude. This force directs the particle towards the strong or the weak field regions, depending upon whether Re(f_{CM}) is positive or negative (FIG. 2a). This is the conventional DEP term (Huang et al., 1992; Jones and Kallio, 1979).

(ii) the right hand term relates to the imaginary (out-of-phase) component of the induced dipole moment and to the field spatial nonuniformity (∇φₓ, ∇φ_{y} and ∇φ_{z}) of the field phase. Depending on the polarity of Im(f_{CM}), this force directs the particle towards regions where the phases of the field component are larger (Im(f_{CM})>0) or smaller (Im(f_{CM})<0) (FIG. 2b). Under the constraint conditions for a travelling electric field, Eq. 4 is reduced to the twDEP force expression i(Huang et al, 1993).

Eq. 4 shows that the force experienced by a particle in an AC electric field arises not only from the field magnitude inhomogeneity as envisioned by Pohl (1978) but also from the field phase nonuniformity. The inventors have termed the particle motion caused by both magnitude and phase nonuniformities generalized dielectrophoresis. Since all field-induced cell motions are understandable in terms of Eq. 4, sophisticated field configurations having both phase and magnitude nonuniformities can be explored by methodology known in the art.

Characterization Of Engineered Microparticles By Rot

The dielectric properties of particles, including engineered microparticles, may be established by electrorotation. In electrorotation, particles are subjected to a rotating electric field and induced to rotate about an essentially stable axis. The method is suitable over other characterization methods as it is relatively straightforward, offers good reproducibility, and provides a means to characterize individual particles. While particle dielectric properties can be probed non-invasively by any of the electrokinetic phenomena, ROT offers the significant advantage that it induces particle rotation about an axis that, for most purposes, can be considered stationary in space. Thus, the particle remains in a position of constant field strength. The ROT torque, \vec{Γ}(t), depends (Arnold and Zimmermann, 1982 and 1988; Fuhr, 1985) not on the inhomogeneity of the electrical field, but on the cross product

\vec{Γ}(t)=−\vec{m}(t)×\vec{E}(t) (5)

and in the frequency domain the magnitude of this torque (Arnold and Zimmermann, 1982 and 1988; Fuhr, 1985) can be written

Γ(ω)=−4πεₘr³Im(f_{CM})E². (6)

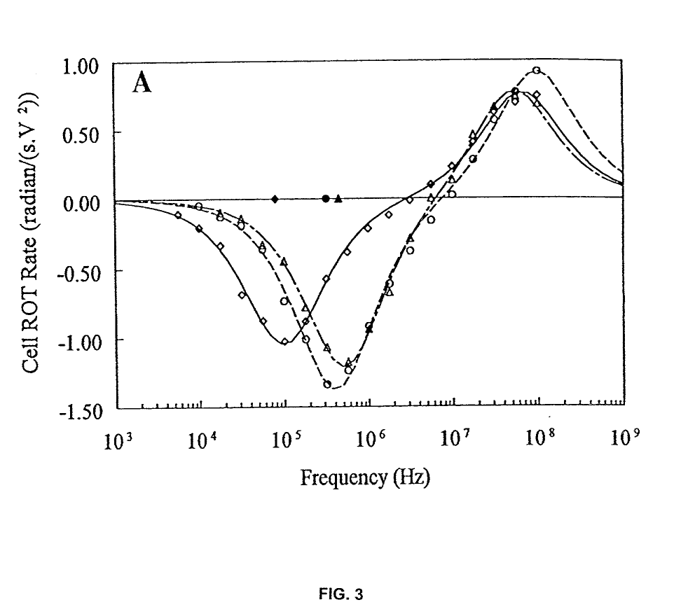

Typical ROT spectra derived for three different particle, or in this case cell, types are shown in FIG. 3. Equation 6 shows that the shape of each spectrum reflects Im(f_{CM}) for each particle type. By applying an appropriate dielectric model, it is possible to derive the dielectric properties of engineered microparticles directly from their ROT spectra. Explicit dielectric modeling of particle properties is most frequently undertaken using dielectric shell models (Huang et al., 1992; Fuhr, 1985; Irimajri et al., 1979), and the inventors (Huang et al., 1992; Wang et al, 1994c; Gascoyne et al, 1994b) and others (Holzel and Lamprecht, 1992) have contributed to the theory that allows dielectric data for particles to be derived from ROT.

The inventors have also analyzed the accuracy with which dielectric parameters can be derived from ROT analyses (Gascoyne et al., 1994b). This allows us not only to understand essential dielectric and structural aspects of the microparticles but also, in conjunction with Eq. 4, to predict the microparticle electrokinetic behavior under all suspension conditions for all electrical field configurations. Analogous magnetic rotation experiments may also be conducted to characterize the microparticle magnetic properties.

Dielectric Modeling of Engineered Microparticles

The structure of an engineered microparticle such as that depicted in FIG. 1 and explained in accompanying text may be approximated, in terms of dielectric properties, by a spherical conductive interior (even if that interior, in turn, includes a dielectric core surrounded by a conductive shell) surrounded by a thin, poorly conducting shell. The complex permittivity εₚ^{*} of such a particle is given (Irimajari et al., 1979; Huang et al., 1992) by

$${ϵ}_{p}^{*} = \{ \frac{{( \frac{r}{r - d} )}^{3} + 2 ( \frac{{ϵ}_{interior}^{*} - {ϵ}_{shell}^{*}}{{ϵ}_{interior}^{*} + 2 {ϵ}_{shell}^{*}} )}{{( \frac{r}{r - d} )}^{3} - ( \frac{{ϵ}_{interior}^{*} - {ϵ}_{shell}^{*}}{{ϵ}_{interior}^{*} + 2 {ϵ}_{shell}^{*}} )} \} \qquad ( 7 )$$

where εᵢₙₜₑᵣᵢₒᵣ^{*} and εₛₕₑₗₗ^{*} are the complex permittivities conductivity and permitivities of the particle interior and the insulating shell, r is the particle radius and d is the thickness of the insulating layer. The cDEP and twDEP AC electrokinetic response of an engineered microparticle may be modeled in accordance with methodology known in the art using Eqs. 4 and 7.

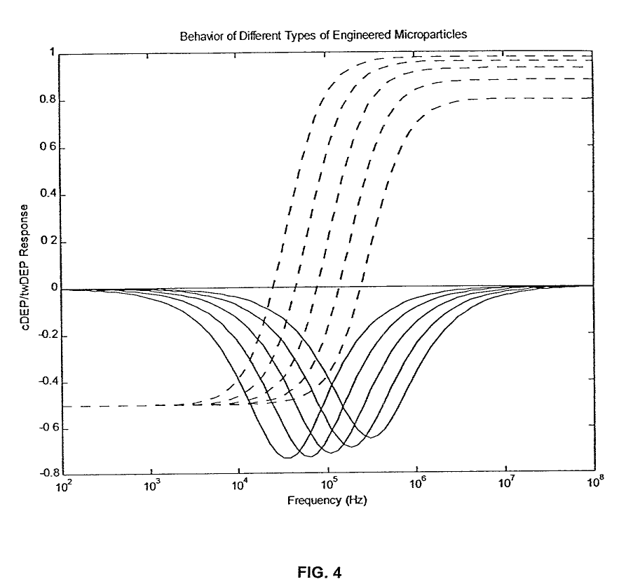

FIG. 4 illustrates the cDEP and twDEP response of engineered microparticles with shell thickness varying between about 1 and 10 nm. It is evident from FIG. 4 that engineered microparticles of different compositions exhibit substantially different responses to AC electrical fields of various frequencies. The frequency response of a single microparticle type is referred to as a dielectric fingerprint and allows discrimination between microparticles having different structures.

Engineered microparticles with more complex dielectric fingerprints may be produced by applying multiple layers of materials of controlled thicknesses over a core material or by using a core material that is dispersive. The AC electrokinetic response of these more complex engineered microparticles may be predicted through the use of a multi-shell dielectric model known in the art, such as that described by Jones (1995), and incorporated herein by reference. In addition, other non-concentric structures may be produced and modeled by a spherical-shell equivalent.

A library of engineered microparticles with different dielectric fingerprints may be readily assembled by producing engineered microparticles with different physical compositions and structures. Microparticles having unique dielectric fingerprints may be individually addressable and may be used as frequency dependent handles to manipulate several different analytes in a sample mixture.

Magnetophoresis

A particle of volume v and magnetic permeability μₚ^{*} placed into an inhomogeneous magnetic field will experience a magnetophoretic force

F_{MAP}=2πμₛR³k_{cm}(μₛ^{*},μₚ^{*},ω_{H})∇H(x,y,z)² (8)

where, μₛ is the magnetic permeability of the suspending medium, R is the radius of the particle, k_{cm}(μₛ^{*},μₚ^{*},ω_{H}) is the Magnetic Clausius-Mossotti factor describing the magnetic polarizability of the particle with respect to its suspending medium, and ∇H(x,y,z)² is the gradient of the square of the magnetic field strength. Here ω_{H} is the frequency of the applied magnetic field and will have the value zero for a static field. In analogy with the dielectric equation (Eq. 2), μₛ^{*} and μₚ^{*} are the complex permeabilities of the suspending medium and particle, respectively. In the case of a static magnetic field, these reduce to the real, static magnetic permeability parameters μₛ and μₚ, respectively.

Note that equation 8 is the magnetic analog of equation 2. Alternatively, if the particle has a permanent volume magnetization m, then the magnetophoretic force will be

F_{MAP}=μₛR³m∇·H(x,y,z)² (9)

It is possible for a particle to have both permanent and inducible magnetic polarization, components. In that case a combination of equations 8 and 9 may apply. For example, a particle may have a high permeability and at the same time demonstrate magnetic remnance. For a formal discussion of magnetophoresis, the reader is referred to Jones (1995).

The use of magnetophoresis to collect magnetically susceptible microparticles is well known in the art. Products from sources such Dynal, Inc. (Lake Success, N.Y.) and Miltenyi Biotec (Auburn, Calif.) are routinely used for magnetophoresis-based separation techniques known as immunomagnetic separation (IMS) and magnetically activated cell sorting (MACS).

Although magnetic microparticles are readily available from many sources, the simultaneous exploitation of magnetic and dielectric microparticle properties for enhanced separations is a novel approach. The device used to discriminate, manipulate and/or isolate engineered microparticles contains both AC electrokinetic and magnetophoretic elements. For example, AC electrokinetic manipulation of engineered microparticles may include cDEP, twDEP, gDEP and ROT using an electrode array to which AC signals are switched. Magnetophoretic manipulation of engineered microparticles may be performed using a strong magnet fitted with a means for providing local magnetic field inhomogeneity in the vicinity of the electrode array. In such a device engineered microparticles experience both AC electrokinetic and magnetophoretic manipulating forces; both the dielectric and magnetic properties of the microparticles may thereby be exploited simultaneously to provide enhanced discrimination and manipulation capabilities.

The Sensitivity of DEP-FFF to Changes in Particle Dielectric Properties

The inventors have shown both theoretically and experimentally (Wang et al., 1998) that for a parallel electrode geometry, the dielectrophoretic levitation force for a dielectric particle suspended in a fluid medium falls off exponentially with height above the electrode. If the particle has a density such that it tends to sediment towards the electrode plane, a stable dielectrophoretic levitation will occur at an equilibrium height given by

$${h}_{eq} = \frac{d}{2 π} ln \{ \frac{3 {ϵ}_{m} {U}^{2} Ap}{2 ( {ρ}_{c} - {ρ}_{m} ) g} Re ( {f}_{CM} ) \} . \qquad ( 1 )$$

Here U is the electrical potential applied to the electrode array, A is a geometrical term, p is the proportion of the applied field that is unscreened by electrode polarization, εₘ is the dielectric permittivity of the suspending medium, (ρ_{c}−ρₘ)g is the sedimentation term, and dielectric polarization occurring at the particle-medium interface is described by the real part of the so-called Claussius-Mossotti factor Re(f_{CM}).

In DEP-FFF, the levitation occurs within a fluid that is flowing in a thin chamber according to a hydrodynamic flow profile. Thus the velocity of the particle will depend upon its levitation height h_{eq} in the flow stream and particles having different values of h_{eq} may consequently be separated from a starting mixture as they flow at differential velocities along the length of the chamber in which the levitation occurs.

In order to appreciate the dependency of a particle's velocity on small changes in its dielectric properties in a DEP-FFF experiment, it is helpful to write it first as a function of the equilibrium height prior to dielectric changes. Differentiating equation (1) with respect to the dielectric properties, we obtain

$$\partial {h}_{eq} = \frac{d}{4 π} \cdot \frac{\partial Re ( {f}_{CM} )}{Re ( {f}_{CM} )} . \qquad ( 2 )$$

If the hydrodynamic flow profile in the separation chamber is parabolic, then the particle velocity will depend on its levitation height according to

$${v}_{p} = 6 ⟨ v ⟩ \frac{{h}_{eq}}{H} ( 1 - \frac{{h}_{eq}}{H} ) \qquad ( 3 )$$

where H is the chamber thickness and <v> is the mean fluid velocity.

Thus we can write the incremental changes in velocity corresponding to incremental changes in height as

$$\partial {v}_{p} = 6 ⟨ v ⟩ ⌈ \frac{1}{H} - \frac{2 {h}_{eq}}{{H}^{2}} ⌉ \partial {h}_{eq} . \qquad ( 4 )$$

We can further substitute from equation (3) for 6<v> to obtain

$$\partial {h}_{eq} = {h}_{eq} \{ \frac{H - {h}_{eq}}{H - 2 {h}_{eq}} \} \cdot \frac{\partial {v}_{p}}{{v}_{p}} \qquad ( 5 )$$

and then relate incremental changes in particle dielectric properties to corresponding changes in the particle velocity, using equation (2), as

$$\begin{matrix}\frac{\partial {v}_{p}}{{v}_{p}} = \frac{d}{4 π {h}_{eq}} \cdot \{ \frac{H - 2 {h}_{eq}}{H - {h}_{eq}} \} \cdot \frac{\partial Re ( {f}_{CM} )}{Re ( {f}_{CM} )} \\ where , once again , & ( 6 ) \\ {h}_{eq} = \frac{d}{2 π} ln \{ \frac{3 {ϵ}_{m} {U}^{2} Ap}{2 ( {ρ}_{c} - {ρ}_{m} ) g} Re ( {f}_{CM} ) \} . & ( 1 )\end{matrix}$$

Inspection of equation (6) reveals that the sensitivity of the particle velocity to incremental changes in height is largest when h_{eq} and Re(f_{CM}) are both small. The extreme sensitivity of the DEP-FFF phenomenon derives from the fact that these two conditions tend to be mutual. The inventors note, for example, from equation (1) that there is a threshold condition for levitation to occur at all,

$$\frac{3 {ϵ}_{m} {U}^{2} Ap}{2 ( {ρ}_{c} - {ρ}_{m c} ) g} \cdot Re ( {f}_{CM} ) > 1$$

This asserts that the dielectrophoretic force must exceed the sedimentation force at the electrode plane for levitation. Clearly, for any prevailing conditions in which

$$\frac{A}{( {ρ}_{c} - {ρ}_{m c} ) g} \cdot Re ( {f}_{CM} ) > 0 ,$$

the applied voltage U can be chosen in order to assure that levitation does indeed occur but that it is nevertheless small. We also notice that smaller magnitudes of Re(f_{CM}) also assure smaller levitation heights. Of course there will be a practical limit to how large U can be and therefore how small Re(f_{CM}) can be in a real application.

Because h_{eq} and Re(f_{CM}) dominate the denominator of equation (6), the sensitivity of the DEP-FFF incremental velocity to changes in the particle dielectric properties can therefore be very large if the appropriate conditions are chosen. In practice, changes in Re(f_{CM}) as small as −0.0001 may be detectable—a truly astonishing sensitivity.

To illustrate this, we observe that in experiments conducted on human HL-60 leukemia cells on an electrode array having 20 micron electrode widths and spacings, the following parameters obtained:

A=−2.77×10¹⁴m⁻³, d=80×10⁻⁶m, p=1, ρ_{c}=1089kg·m⁻³,

ρₘ=1033 kg·m⁻³, εₘ=78·εₘ and H=200×10⁻⁶m.

From equation (1), the levitation height is calculated as

h_{eq}=6·37×10⁻⁶ In(−522Re(f_{CM})),

and the velocity sensitivity parameter is

$$\frac{\partial {v}_{p}}{{v}_{p}} = \frac{6 \cdot 37 \times {10}^{- 6}}{{h}_{eq}} \cdot \{ \frac{2 \times {10}^{- 4} - 2 {h}_{eq}}{2 \times {10}^{- 4} - {h}_{eq}} \} \cdot \frac{\partial Re ( {f}_{CM} )}{Re ( {f}_{CM} )} .$$

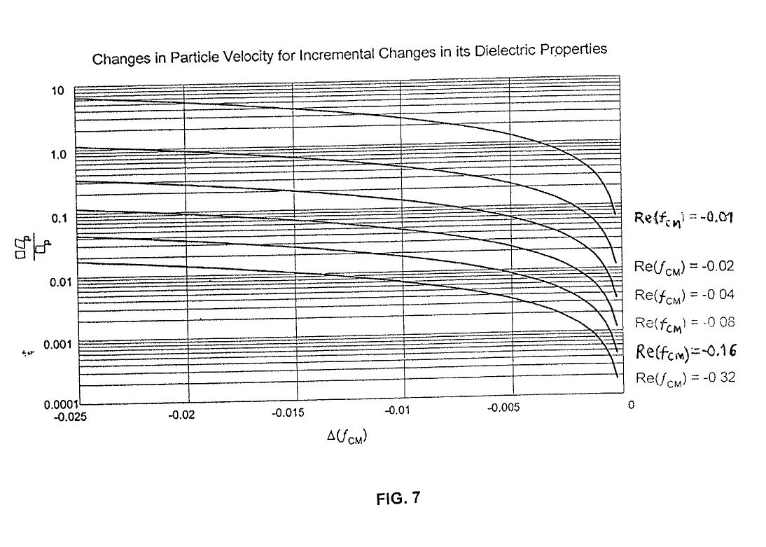

FIG. 7 shows, for these experimentally-verified parameters, the dependency of the particle velocity sensitivity, expressed as a % change in particle velocity, on small changes in Re(f_{CM}) for starting values of −0.01, −0.02, −0.04, −0.08, −0.16 and −0.32. We note that an increment in the magnitude of Re(f_{CM}) of 0.025, the maximum shown in the figure, is still considered to be extremely small.

FIG. 7 shows that if Re(f_{CM}) of the particle/medium combination is small then the sensitivity of the DEP-FFF velocity to changes in dielectric properties is very large indeed. For example, for the starting value Re(f_{CM})=−0.01, a change in Re(f_{CM}) of −0.005 produces a 120% increase in the particle velocity (magenta curve). We can reliably measure changes in particle velocity as small as 2%, so the sensitivity is sufficient to detect a change in Re(f_{CM}) of only −0.0001. On the other hand if the starting value of Re(f_{CM}) is −0.32, detection of changes in the particle velocity will be reliable only for increments in Re(f_{CM}) larger than about −0.03. For the highest level of sensitivity in a DEP-FFF detection assay, the dielectric particles and suspending medium should clearly be chosen so that Re(f_{CM})→0. We shall consider now, therefore, the conditions under which this can be achieved.

The Claussius-Mossotti Factor f_{CM}

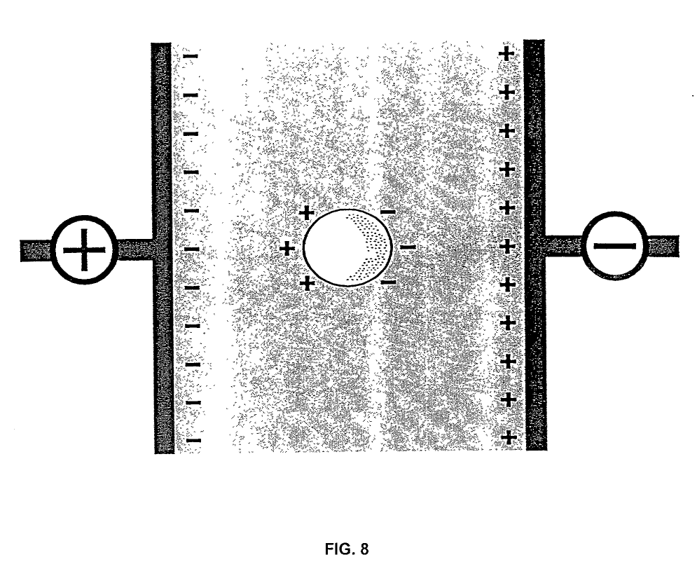

Consider a particle placed inside a dielectric medium to which an electric field has been applied. The dielectric suspending medium and particle will polarize in response to the field. However, if the dielectric properties of the particle are dissimilar from those of the suspending medium then the particle and medium will exhibit dissimilar degrees of polarization and the interface between the two will undergo a local polarization to ensure that the dielectric displacement D across the interface is continuous. In FIG. 8, the oval particle has a low dielectric permittivity and does not polarize appreciably. On the other hand the supporting medium, which has a high dielectric permittivity, polarizes and an interfacial polarization at the particle/medium interface arises to maintain continuity in dielectric displacement. This represents fairly accurately the case of an air bubble in aqueous suspension. It also represents a simple- minded view of a polymer bead suspended in water in a DEP-FFF experiment (but see later for very important discrepancies). The combination of all of the dielectric polarizations determines the dielectrophoretic response of the particle if the applied electrical field is inhomogeneous. The Claussius-Mossotti factor f_{CM} expresses the overall polarizability of the particle in the suspending medium and f_{CM} therefore includes polarization terms for both the particle and the medium. A central problem of AC electrokinetics is determining f_{CM} for a given particle and medium combination so that the dielectrophoretic forces can be modeled.

For a spherical particle, the Claussius-Mossotti factor is given by

$${f}_{CM} = ( \frac{{ϵ}_{p} - {ϵ}_{m}}{{ϵ}_{p} + 2 {ϵ}_{m}} )$$

where εₚ is the effective permittivity of the particle and 68 ₘ is that of the medium. In the figure, the particle shown is non-polarizable so that εₚ<<εₘ. In this case f_{CM·≈-}0.5 so that Re(f_{CM})≈−0.5 also. In a dielectrophoretic experiment, this corresponds to the condition for maximum negative dielectrophoresis. Substituting this value for Re(f_{CM}) into equation (1) and (6) gives shows that a huge change in the dielectric properties of the particle εₚ would be required in order to create a substantial change in particle velocity under these conditions and one concludes that DEP-FFF would not be very sensitive for detecting alterations in particle dielectric properties. On the other hand we saw earlier that the DEP-FFF sensitivity increases as Re(f_{CM})→0. This implies that

$$Re ( \frac{{ϵ}_{p} - {ϵ}_{m}}{{ϵ}_{p} + 2 {ϵ}_{m}} ) \to 0$$

Optimizing DEP-FFF for detecting small responses in the dielectric properties of particles to target agents therefore boils down to exploring the conditions under which the particles and their suspending medium have very nearly the same effective relative permittivities.

The Claussius-Mossotti factor for Engineered Microparticles

Note that εₚ is the effective particle permittivity. In the case of an air bubble, εₚ could be modeled as a constant equal to the permittivity of free space. However, depending upon the structure and composition of the particle type in question, the expression for εₚ and its corresponding dielectric model may be rather complex. For example, a simple model for an engineered microparticle such as that shown in FIG. 3, εₚ as arises from a concentric shell system composed of a conductive interior having a permittivity ε_{c}, and a conductivity σ_{c} surrounded by a very thin insulating layer having a permittivity εₛ and a conductivity σₛ.

The corresponding effective permittivity of this so-called shell system is very frequency dependent. It is this frequency dependency that can be very effectively exploited to allows microparticles of different types to be characterized, discriminated and sorted by AC electrokinetic methods. Furthermore, appropriate suspending medium permittivity εₘ, conductivity σₘ, and applied frequency conditions can readily be selected so that the effective permittivity of an engineered microparticle type is very close to that of the suspending medium. In this way, microparticles lend themselves to high discrimination by DEP-FFF.

For engineered microparticles modeled after mammalian cells, the shell conductivity σₛ will be extremely small and its influence in comparison to capacitance effects from εₛ can be neglected. Conversely, for the frequency range, from 10 kHz to <1 MHz where capacitance effects are important, the influence of the conductivity of the suspending medium σₘ is much greater than that of its permittivity εₘ. Under these conditions, the real part of the Claussius-Mossotti factor for engineered microparticles modeled after mammalian cells can be approximated as

$$Re ( {f}_{CM} ) = \{ \frac{1}{{( {σ}_{m} / 2 π f )}^{2}} - \frac{2}{{( rC )}^{2}} \}$$

Where C is the specific membrane capacitance of the microparticle in F/m², r is the radius of the microparticle and f is the frequency of the applied field. We note that in this case the DEP crossover condition, where Re(f_{CM})→0, occurs when

$$f = \frac{1}{\sqrt{2 π}} \cdot \frac{{σ}_{m}}{rC}$$

The fundamental condition that has to be satisfied in order to allow for the discrimination of two different types of microparticles by DEP-FFF is that they be levitated to different heights. When the above approximation for the real part of the Claussius-Mossotti factor is valid, this condition can be expressed, using equation (1), as

$$Δ H = \frac{d}{4 π} ln \{ \frac{\frac{1}{{( {σ}_{m1} / 2 π f )}^{2}} - \frac{2}{{( {r}_{1} {C}_{1} )}^{2}}}{\frac{1}{{( {σ}_{m2} / 2 π f )}^{2}} - \frac{2}{{( {r}_{2} {C}_{2} )}^{2}}} \cdot \frac{( {ρ}_{p2} - {ρ}_{m} )}{( {ρ}_{p1} - {ρ}_{m} )} \} \neq 0$$

where the subscripts 1 and 2 refer, respectively, to the two microparticles to be separated. It follows that

$$\frac{1}{{( {σ}_{m1} / 2 π f )}^{2}} - \frac{2}{{( {r}_{1} {C}_{1} )}^{2}} ( {ρ}_{p2} - {ρ}_{m} ) \neq \frac{1}{{( {σ}_{m2} / 2 π f )}^{2}} - \frac{2}{{( {r}_{2} {C}_{2} )}^{2}} ( {ρ}_{p1} - {ρ}_{m} )$$

which can be satisfied in three ways, namely (1 and 2) if either of the two DEP crossover frequency terms defined within the square brackets approaches zero while the other is non-zero, a condition that can always be satisfied if

r₁C₁≠r₂C₂

or (3) when the frequency is far below either crossover frequency if

(ρₚ₁−ρₘ)≠(ρₚ₂−ρₘ)

In this third case, microparticle size, density and shell capacitance all combine as factors to determine microparticle separability

### EXAMPLES

The following examples are included to demonstrate specific embodiments of the present disclosure. It should be appreciated by those of skill in the art that the techniques disclosed in the examples which follow represent techniques discovered by the inventor to function well in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the invention.

### Example 1

### Engineered Microparticle Design Considerations

Life science research typically requires analysis of particles that range in size from about 100 nm to 10 μm in diameter. The main forces acting on particles in this size range are sedimentation forces and randomizing forces due to Brownian motion. For a particle of radius 1 μm and density of 1.05 g/cm³ suspended in aqueous medium (ρ=1.00 g/cm³) at 25° C. the sedimentation and Brownian forces each have magnitude of approximately 2×10⁻¹⁵ N.

To effectively use conventional dielectrophoresis as a manipulating force, the cDEP force must be greater than the other forces acting on the particle, and in one embodiment, about an order of magnitude greater than the other forces acting on the particle. According to Eq. 4, if Re(f_{CM})=0.5, then ∇E(rms)² should be approximately 9×10¹²V²/m³ to give a cDEP force that is ten times greater than the sedimentation or Brownian forces (Pething and Markx, 1997).

Using microelectrodes, and methodology known in the art, it is possible to generate fields of this magnitude with an applied voltage of about 10 V or less. The sedimentation and conventional dielectrophoretic forces are both proportional to r³, while the randomizing forces of Brownian motion are proportional to r⁻¹. For particles larger than 1 μm, the sedimentation and cDEP forces are the dominant forces acting on particles, and conventional dielectrophoresis may be used to manipulate particles about 10 μm in diameter or larger. For smaller particles, Brownian motion forces dominate over sedimentation forces. By using electrodes with submicron geometries, one may generate cDEP forces capable of manipulating viruses and other particles that are about 100 nm in diameter or smaller (Muller et al., 1996).

With the benefit of this disclosure, engineered microparticles may be designed with properties that make them amenable to AC electrokinetic and magnetophoretic manipulation. As discussed previously for the specific case of cDEP manipulation of particles, the magnitude of the cDEP force must be sufficient to overcome the influence of the other forces acting on the particles in the system. This is true for any AC electrokinetic or magnetophoretic manipulation. The forces acting on particles are generally sedimentation forces and Brownian motion induced randomizing forces. Since the magnitude of these forces depends upon the particle properties, the engineered microparticles may be designed so that the effect of the AC electrokinetic force is maximized by appropriately scaling the influence of competing forces.

Sedimentation forces may be scaled, for example, by producing engineered microparticles with an effective density between about 1.0 and 2.0 g/cm³. A small (<1.5×10⁻¹² N) sedimentation force will then act on 10 μm particles of this density when they are suspended in an aqueous medium. For most applications this characteristic is preferred, as microfabricated AC electrokinetic devices are typically designed with microelectrodes on the lower surface of the device. The electric field strength, and therefore the AC electrokinetic force, is most pronounced near the electrode plane.

Negative buoyancy may be used to ensure that the microparticles fall to the electrode plane where they can be held by positive cDEP or levitated by negative cDEP. The Brownian forces may be reduced by designing microparticles that are about 10-20 μm in diameter. The influence of Brownian forces on particles of this size is negligible when compared to the magnitude of the DEP forces that are typically used for the manipulation of engineered microparticles. Analogous arguments are applicable to the design of the magnetic microparticle properties and the magnetic field.

One approach to achieve a final effective density in a specified range may be as follows: custom engineered microparticles may be produced by thin-film deposition of conductive, insulating and/or magnetically susceptible materials on low density (<1.3 g/cm³) spherical substrates. It is known from Maxwell's laws of electromagnetism that a conductive spherical shell is indistinguishable from a solid conducting sphere in response to an externally applied electrical field. The conductive layer may be a thin-film of metal such as gold, silver, platinum or copper about 10-100 nm thick.

This layer may be applied over the substrate by physical vapor deposition (PVD) or electroless plating (Elshabini and Barlow, 1998) according to principles known in the art, to form the conductive core. The insulating material may be a thin film of metal oxide such as Al₂O₃ or polymer material such as polystyrene or PTFE. Such materials may be applied through PVD or microencapsulation (Lim, 1984) techniques, for example.

Since the densities of the conductive and insulating layers may range from 8.9-21.4 g/cm³ and 1.1-2.2 g/cm³ the corresponding substrate must have a low density to yield a finished microparticle in the desired density range. Polystyrene and hollow glass microparticles about 10-1000 μm in diameter are commercially available with a density of approximately 1.0 g/cm³ from several companies including Dynal, Inc. (Lake Success, N.Y.), Miltenyi Biotec (Auburn, Calif.), Cortex Biochem, Inc. (San Leandro, Calif.), and BioSource International (Camarillo, Calif.).

Similar considerations apply to the design of microparticle magnetic properties such that appropriate microparticle density may be achieved. Suitable magnetic materials for microparticle core or layer construction include ferrites, rare-earth containing ceramics and glasses, as well as iron, cobalt, titanium and other materials containing atoms or molecules with uncompensated electron spins.

Characteristics that determine the dielectric properties of microparticles include their size, surface charge, density, composition and electrical conductivity. With the benefit of this disclosure and technology known in the art, all of these parameters may be modified in order to achieve a desired dielectric fingerprint. In particular, engineered microparticles may be fabricated so as to incorporate defined surface and internal dielectric, magnetic, and density properties through the use coatings, internal and surface layers and internal compartments and/or cores, each of which may be dielectric, conductive, magnetic or non-magnetic. The dielectric and electrical properties of the microparticle surface and of each coating, layer, compartment and core may be different. The overall dielectric and magnetic properties of a microparticle will be determined by the synergistic dielectric and electrical contributions of each of its component parts and by the presence in them of magnetically susceptible materials. By combining structural elements having appropriate dielectric and electrical properties, different types of microparticles may be synthesized that have distinct and distinguishable dielectric properties.

The physics of dielectrics and magnetics makes it possible to manufacture, in more than one way, microparticles having essentially similar dielectric and/or magnetic properties. For the purpose of this invention, all microparticles having similar dielectric and magnetic properties in a defined frequency range of interest shall be considered as being identical microparticles even if their underlying physical compositions are different.

Examples of microparticle structures include simple spheres of latex; metal; glass; semiconductors; plastic or magnetic materials, with or without controlled surface properties or coatings; carbon composites and other materials known in the art for the manufacture of conductive or resistive components; silicon; germanium; selenium; and/or gallium-arsenide or other elemental or compound materials known in the art for their semiconducting properties, whether doped or undoped by trace agents to modify their conductive or dielectric properties.

More complex structures include microparticles having one or more of the following features: (i) an electrically non-conductive membrane-like coating with an electrically more conductive interior; (ii) a layer or core containing a dielectric material having a dielectric dispersion within a frequency range of interest; (iii) a highly conductive surface layer, or core; (iv) a surface with a net charge that contributes to the properties of the microparticle through interaction with a dielectric medium; (v) one or more layers or a core possessing magnetic susceptibility.

The present disclosure concerns any microparticle type whose overall dielectric and magnetic properties are specifically chosen such that the microparticles may be used for isolation, identification, characterization, or other manipulation of target analytes through AC electrokinetic or combined AC electrokinetic and magnetic methods.

### Example 2

### Experimental Studies

Silver-coated, hollow glass spheres were obtained from Potters Industries (Valley Forge, Pa.) and custom encapsulated in varying thicknesses of polystyrene by Theis Technology (St. Louis, Mo.) using a surfactant-free microencapsulation protocol. The resulting microparticle structure was similar to that depicted in FIG. 1. Upon application of an inhomogeneous electric field from a castellated, interdigitated electrode array, microparticle manipulation was accomplished by switching the field frequency and voltage. Dielectric responses varied in accordance with the predictions of Eqs. 4 and 7. The results confirm the analysis presented here and indicate that both the dielectric and conductive properties of the polystyrene coating define microparticle behavior as expected. Experiments using dielectric ferrite microparticles from Dynal, Inc. (Lake Success, N.Y.) also confirmed that magnetic and DEP forces may be used simultaneously for microparticle manipulations.

### Example 3

### Applications of Engineered Microparticle Technology

The utility of microparticle-based technologies for the identification, manipulation and isolation of target cells is universal. Recently, the use of microparticles in molecular biology has become widespread and promises to redefine the methodologies employed in life sciences studies wherever cell or molecular targeting or recognition is required. Yet current approaches are one-dimensional and offer little flexibility. For instance, parallel probing of multiple targets is not possible, targets may only be attracted to a collection site so that negative selection (the preference in some sorting applications) is difficult if not impossible, and sorting is essentially digital (targets cannot be discriminated according to binding efficiencies but only according to whether or not they bind any number of microparticles ranging from one to tens of thousands). The methods described here overcome these limitations and offer the potential for separating several targets simultaneously from a mixture, for using both positive and negative selection to greatly enhance the purity of isolated fractions, and for allowing targets to be discriminated according to their binding efficiencies (thus, cells could be sorted according to the number of antibody binding sites on their surfaces rather than just according to whether or not they had any binding sites).

Immediate applications for the engineered microparticle technology include:

1) sorting cells according to the concentration of surface markers including the CD antigens, growth factor receptors, and/or other membrane-associated proteins or moieties;

2) isolation of blood cell subpopulations of high purity;

3) removal of tumor cells and/or T-cells from stem cell harvests;

4) isolation and identification of pathogens from blood, urine and other patient samples;

5) isolation and identification of pathogens in public water supplies and in food preparation and processing facilities;

6) isolation of subcellular compartments such as vesicles and organelles;

7) isolation and identification of nucleic acids, proteins, and other biomolecules from biological samples including those of extremely small volume.

### Example 4

### Engineered Microparticle Technology for Analyte Separation and Identification Based on Cluster of Differentiation, or CD, Antigens Found on the Surface of Cells

Three different microparticle types are engineered such that they each have different cDEP behavior as labeled a, b and c in FIG. 4. By linking a probe for CD3 to engineered microparticles with cDEP behavior labeled a, a probe for CD4 to microparticles with cDEP behavior labeled b, and a probe for CD18 to microparticles with cDEP behavior labeled c, three different engineered microparticle labels may be made. A mixture of these three different labels may then be used to simultaneously label a blood sample containing many different cell subpopulations in a single labeling step.

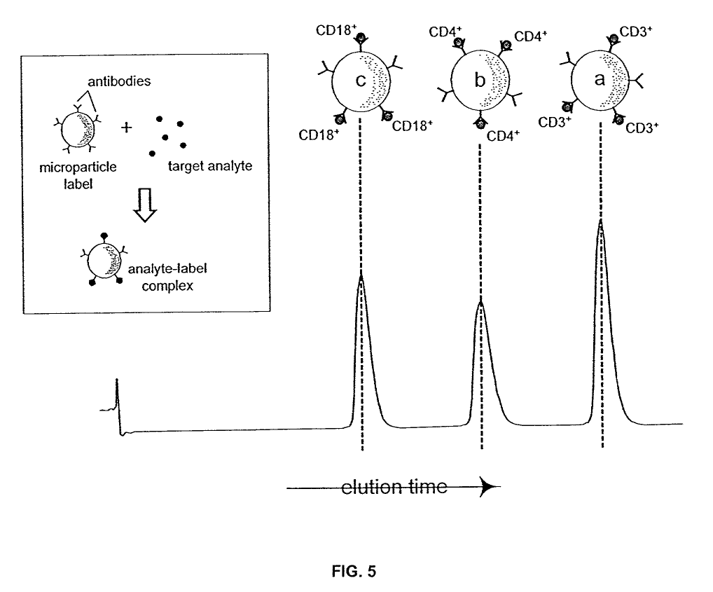

Using an AC electrokinetic-based separation method such as DEP-FFF, the three different microparticle types, and thus CD3⁺, CD4⁺ and CD18⁺ cells, may be separated in a single DEP-FFF separation step (FIG. 5). Using this method, analysis of cell subpopulations not distinguishable from other subpopulations by their size, density or surface specific capacitance characteristics alone is made accessible.

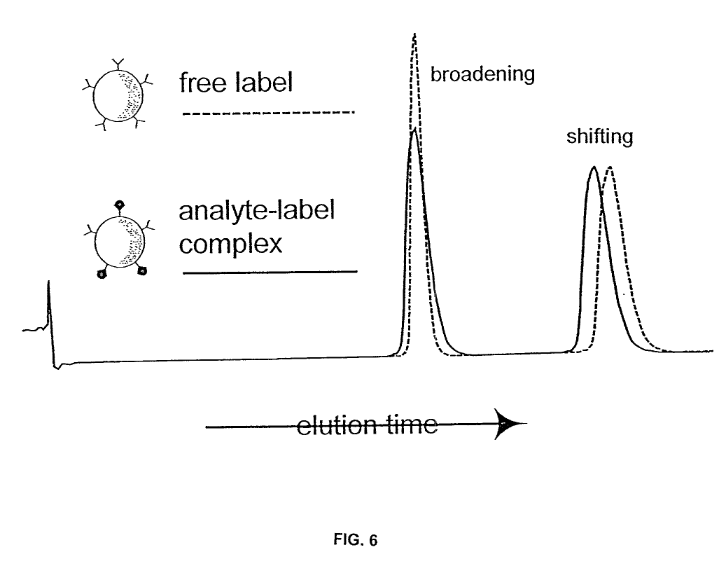

In a DEP-FFF separation, the different microparticle types fractionate into well-defined bands, each of which emerges as a single, well-defined elution peak (FIG. 5). In the case of free, unbound microparticle labels, the peak shape is relatively narrow and sharp. However, because the dielectric properties of the microparticles are perturbed -upon analyte binding, the peak shape of analyte-label complexes is broader and/or exhibits a shift in elution time (FIG. 6). The extent of this perturbation may be dependent upon the nature of the analyte and the extent of analyte binding. It should be noted that such elution peak changes may be used as the basis for quantitative methods of analyte detection.

### Example 5

### Engineered Microparticles

Microparticles may be fabricated using self-assembled monolayers (SAMs) of alkanethiolate on silver or gold metalized hollow glass cores. Alkanethiols CH₃(CH₂)ₙSH chemisorb spontaneously onto gold surfaces to form alkanethiolates: X(CH₂)ₙSH+Au⁰ζX(CH₂)ₙS⁻Au^{I}+½H₂. The alkanethiolates self-organize into densely packed, robust monolayer film. Such films have been extensively characterized, and their insulating properties established. The thickness of an alkanethiol SAM film is dependent upon the number n of methylene groups in the alkyl chain. The dielectric properties of engineered microparticles made with different alkane chain lengths may be investigated. Experiments may be performed with hybrid bilayer membranes formed by the fusion of lipid vesicles with self-assembled alkanethiolate monolayers. The effects of altering the alkanethiol head group X may be determined. In addition other molecules may be adsorbed, such as thiolated DNA to the metalized microparticle surface. Finally, protocols may be developed for linking protein and oligonucleotide capture probes the alkanethiolate head groups.

### Example 6

### Detection of Chemical Biological Warfare Agents

The engineered microparticles discussed herein may be applicable to an immense range of assays extending from detection of CBW agents to detection of medical, chemical, agricultural and environmental analytes. A microparticle-based sandwich assay may be developed to detect specific protein simulants such as cholera toxin-subunit (CTB) and staphylococcal enterotoxin B (SEB). In addition a microparticle-based sandwich assay may be developed for tumor necrosis factor (TNF), an early indicator of a challenged host immune system. Monoclonal antibodies directed against these proteins are available and may be used to construct the capture and labeling probes. Engineered microparticle-based sandwich assays may be developed to detect specific nucleic acid sequences derived from bacterial simulants such as Bacillus subtilis and Escherichia coli serotype O157:H7. Oligonucleotide probes that are complimentary to mRNA sequences found in these organisms are readily obtained. By utilizing dielectrophoresis to focus the analyte-microparticle complexes into a densely packed spherical region, the local analyte concentration may be raised by several orders of magnitude, eliminating the need for nucleic acid amplification, and increasing the assay sensitivity. Existing one-pot assays may be adapted to the PFP platform. Good candidates for adaptation include the bicinchoninic acid (BCA) protein assay from Pierce and the LIVE/DEAD viability/cytotoxicity assay from Molecular Probes.

### Example 7

### Indexing

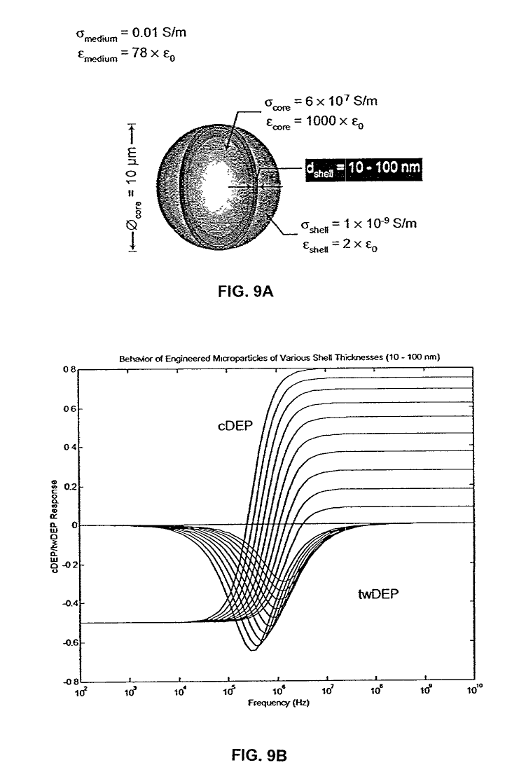

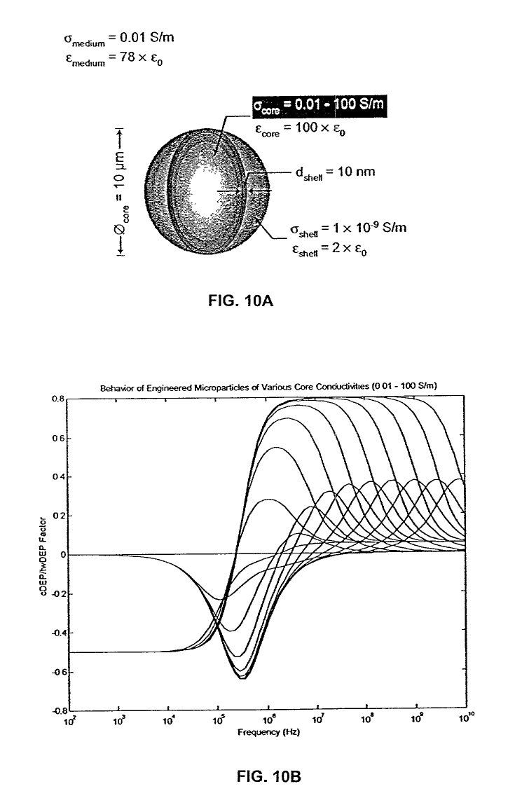

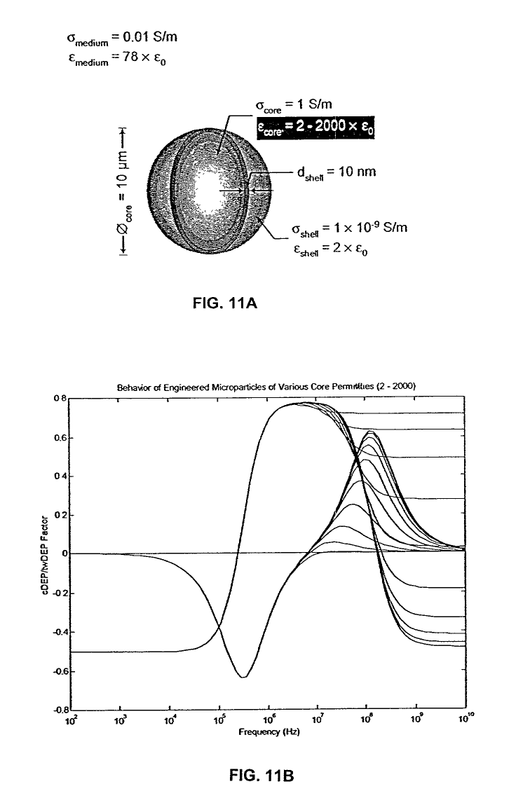

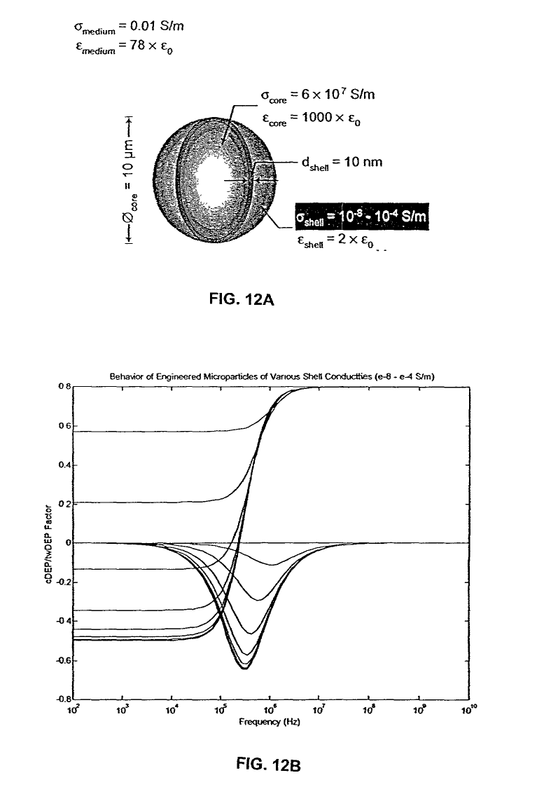

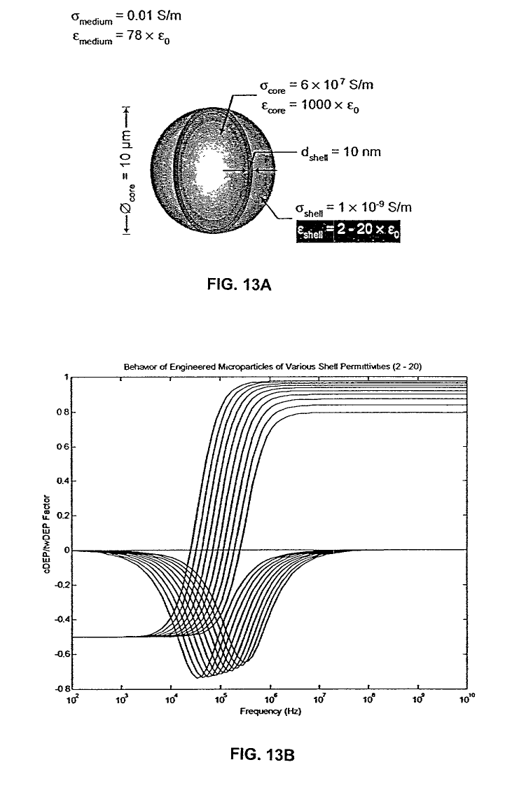

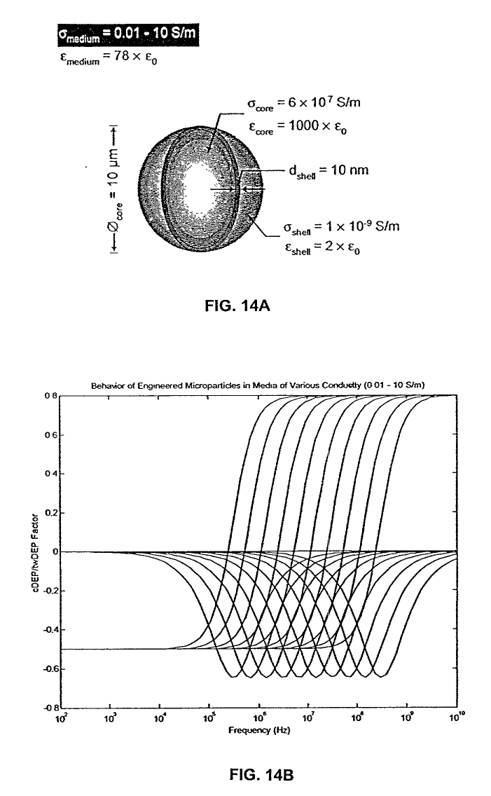

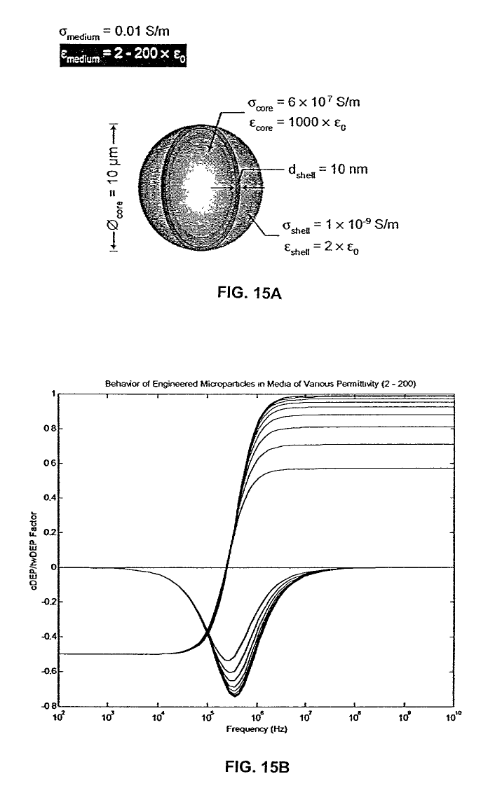

FIGS. 9-15 show engineered microparticles having different dielectric properties. Shown also are the response versus frequency relationship for these particles.

In one embodiment, these particles may be used as an indexing library. In particular, each different microparticle may be made to bind to a different analyte and then simultaneously manipulated, identified, sensed, and detected according to the different response characteristics of the library shown in FIGS. 9B, 10B, 11B, 12B, 13B, 14B, & 15B.

### Example 8

### Sandwich Assays

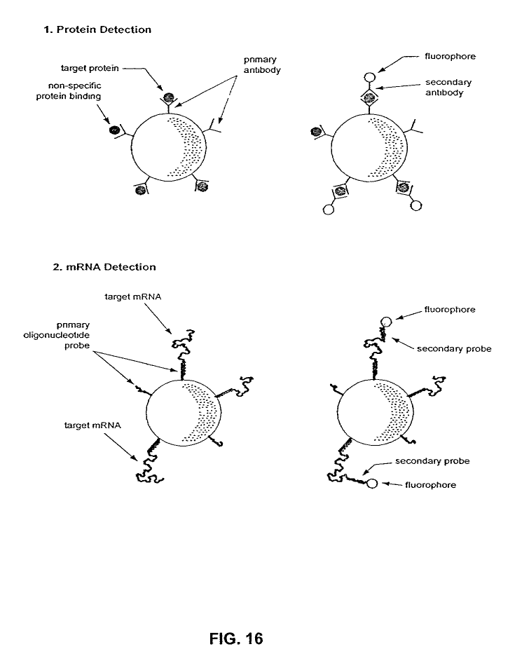

FIG. 16 is a schematic illustrating sandwich (double label) assays that may be used for detecting protein and mRNA in studies in accordance with the present disclosure. As illustrated, the engineered microparticles of FIG. 16 may include linking elements designed to interact with proteins and/or mRNA. Labels, such as fluorophores or bioluminescence labels, may act as secondary probes.

With the benefit of the present disclosure, those having skill in the art will appreciate that the engineered microparticles of FIG. 16, along with the target analytes (and labels) attached thereto may be manipulated using dielectrophoretic forces. In particular, the complexes of FIG. 16 may be sorted, separated, trapped, sorted and generally processed using dielectrophoresis. This processing may take place on a reaction surface such as the surface disclosed in pending U.S. application Ser. No. 09/249,955, filed Feb. 12, 1999, and entitled, “Method And Apparatus for Programmable Fluidic Processing,” which has already been incorporated herein by reference and/or the field-flow fractionation device disclosed in U.S. Pat. No. 5,993,630 which has also been incorporated by reference. A specific example of processing that may be done on a reaction surface is using dielectrophoresis to (a) pull complexes such as those shown in FIG. 16 from a solution, and (b) process those complexes upon the reaction surface.

The formation of the complexes of FIG. 16 may be detected by noting the difference in dielectric properties before and after the formation of the complex. This difference may be measured using one or impedance sensors known in the art or any other methodology known in the art for measuring dielectric, electrical, or physical properties. Plasmon resonance is an example of one such methodology.

According to one embodiment of the present disclosure, different engineered microparticles may be manufactured so that each microparticle has different dielectric properties. For instance, each engineered microparticle may be made with a different thickness and/or composition of its insulating layer(s). Together, this group of microparticles may form a library.

To each different microparticle of the library, a different linking element may be applied. The microparticles may then be admixed with a sample containing one or more target analytes to form one or more complexes. The dielectric properties of each microparticle may be distinguished from the dielectric properties of its corresponding complex using suitable impedance sensors. This distinguishing of dielectric properties allows one to detect if a complex has been formed. Further, the dielectric properties of each microparticle may be distinguished from one another. This distinguishing allows one to determine the identity of the microparticle being detected.

### Example 9

### Engineered Microparticles With One or More Self-Assembled Monolayers

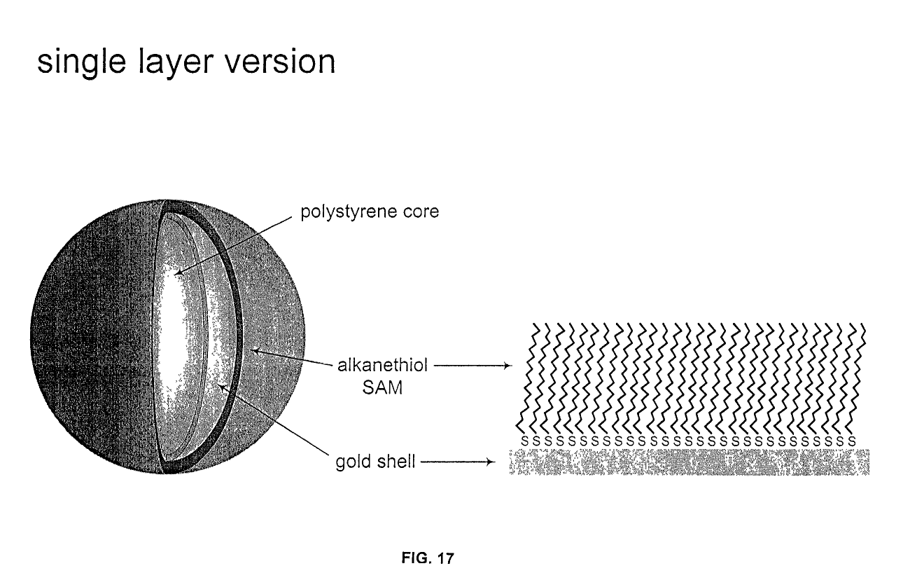

FIGS. 17-21 show multi-layered engineered microparticles according to embodiments of the present disclosure.

FIG. 17 shows a single layer engineered microparticle. It includes a polystyrene core coated with a conductive gold shell. An insulator such as a self-assembled monolayer (an alkanethiol self-assembled monolayer is illustrated) may coat the conductive gold shell. By varying, for example, the size and/or composition of the insulating layer and/or the conductive layer, one may vary the dielectric properties of the engineered microparticle.

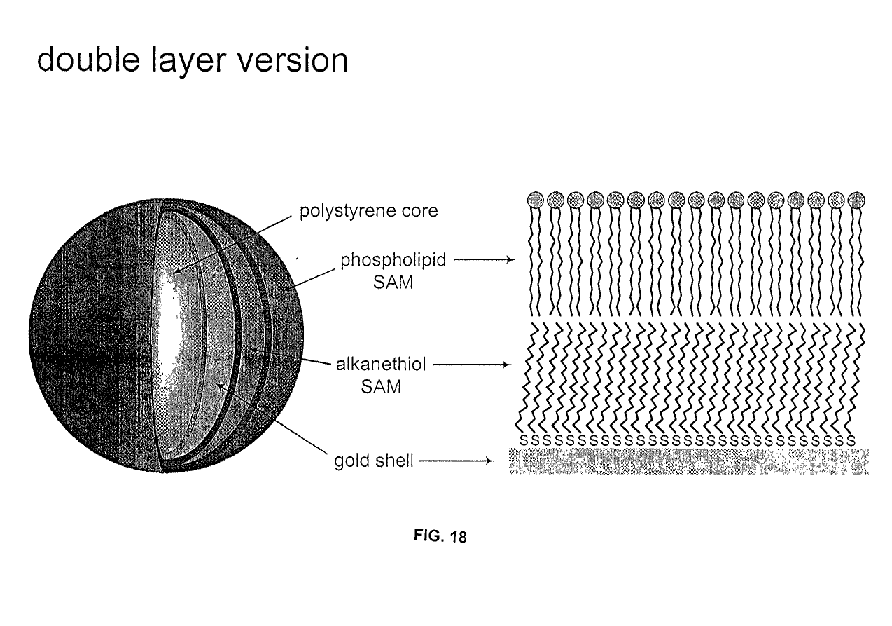

In FIG. 18, a two-layered engineered microparticle is shown. It comprises the following layers: a polystyrene core, a gold shell, an alkanethiol self-assembled monolayer, and a phospholipid self-assembled monolayer.

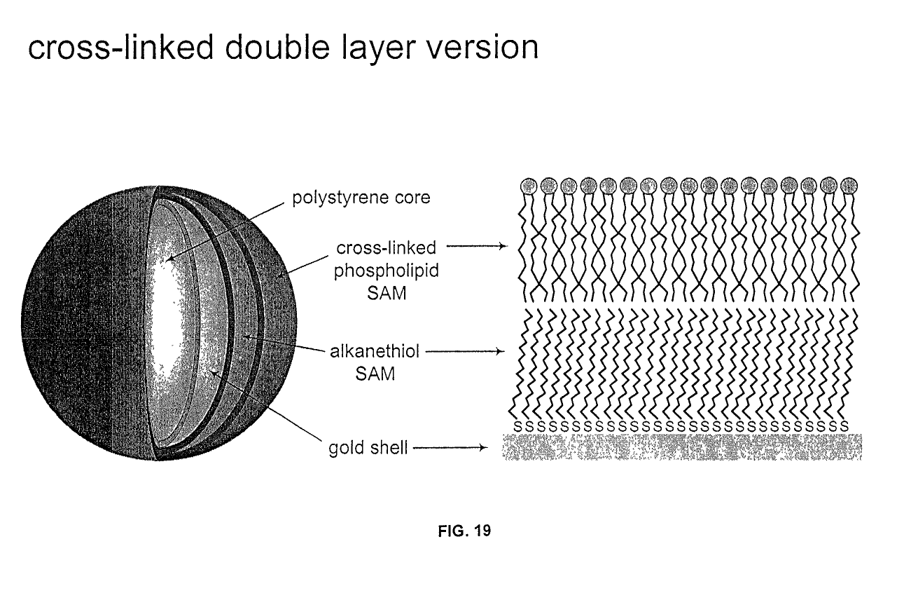

In FIG. 19, the phospholipid self-assembled monolayer is cross-linked. When oxidized, the unsaturated lipids cross-link to form polymer structures. Since it is cross-linked, the phospholipid layer, which has a hydrophilic “head” and hydrophobic “tail,” is more stable in organic solvents.

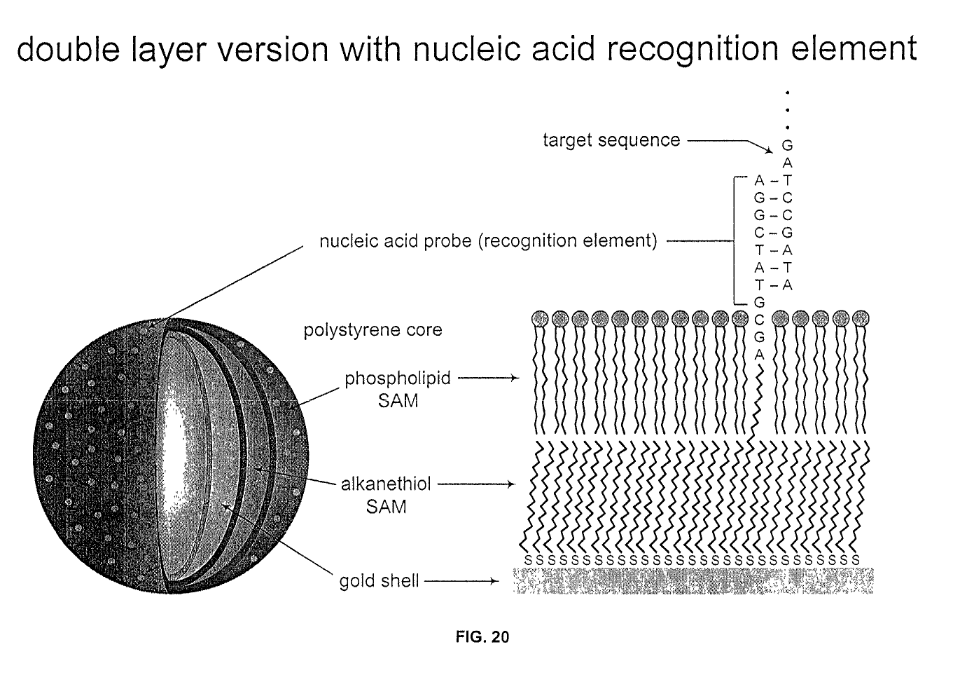

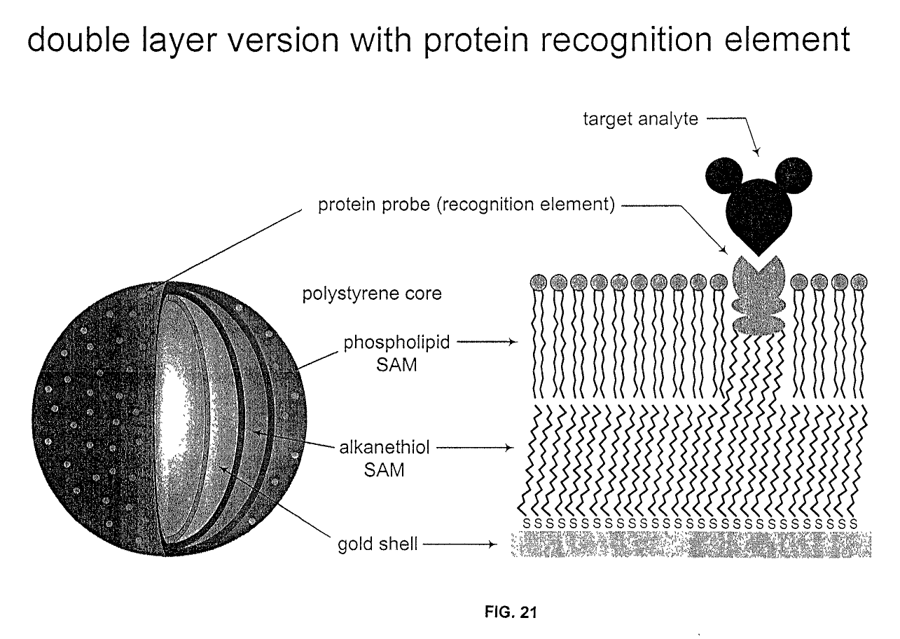

FIGS. 20 and 21 show two-layered engineered microparticles including linking elements. In FIG. 20, the linking element is a nucleic acid probe. In FIG. 21, it is a protein probe. In each of these figures, the linking element is bound to the gold conductive core. However, it will be understood that the linking elements may be bound to the outer or inner insulating layer.

With the benefit of this disclosure, it will be apparent that the microparticles of FIGS. 17-21 may include more or fewer layers. For instance, beyond the gold shell layer (which need not be solid) and the SAM layer(s) shown, one or more additional SAMs or other layers may be added. One or more of the layers may be crosslinked, and one or more labels may be added to the linking elements.

Further, one will understand that the conductive gold shell of FIGS. 17-21 may be substituted with any suitable conductor, including conductive polymers or the like. Additionally, the polystyrene core may be substituted with any other suitable material.

### Example 10

### Fabrication Considerations

The alkanethiolates disclosed herein self-organize reliably into robust, densely packed monolayer films of reproducible thickness. Additionally, biomimetic hybrid bilayer membranes (HBM's) can be formed by fusing phospholipid vesicles with engineered microparticle cores that have already been coated with alkanethiolate monolayers. The thickness of the insulating layer surrounding the core of an engineered dielectric microparticle is dependent on the both the number of methylene groups in the alkyl chain of the alkanethiol SAM film and the number of methylene groups in the lipid tail of the phospholipid used to form the hybrid bilayer membrane.

Therefore, one may produce a library of particles with insulating layers of different thicknesses and different dielectric properties by simply changing the length of the hydrocarbon chain in the alkanethiolate and phospholipid layers of an the engineered microparticles shown herein.

Gold-coated polystyrene microparticles may be obtained from Dynal Biotech that are uniform (coefficient of variation <5%) 9.6 μm in diameter with a density of 2.2 g/cm³. The inventors have constructed four different types of engineered dielectric microparticles by forming self-assembling monolayers of alkanethiolate and phospholipid on the gold-coated polystyrene core particles. Engineered microparticles with a relatively thin insulating layer have been made coating the core particles with a single alkanethiolate monolayer of:

(i) nonyl mercaptan [CH₃(CH₂)₈—SH] to give a C₉ insulating layer,

or

(ii) octadecyl mercaptan [CH₃(CH₂)₁₇—SH] to give a C₁₈ insulating layer.

Engineered microparticles with thicker insulating layers have been made by forming a second insulating monolayer of DMPC [1,2-Dimyristoyl-sn-Glycero-3-Phosphocholine]phospholipid over the alkanethiolate layer as follows:

(iii) nonyl mercaptan plus DMPC to give a C₂₃ insulating layer,

or

(iv) octadecyl mercaptanplus DMPC to give a C₃₂ insulating layer.

Each sample of engineered dielectric microparticles was made by first washing 10 mg of the gold-coated core microparticles in 10 ml of absolute ethanol in a glass tube to clean the gold surface of microparticles. After several minutes of mixing, the microparticles were pelleted by centrifugation using a bench-top centrifuge, the ethanol was decanted off and the washed microparticles were and combined with 10 ml of a 1 mM solution of nonyl or octadecyl mercaptan in absolute ethanol. This suspension was gently mixed for at least 12 hours to ensure the formation of a well-organized, high-integrity self-assembled monolayer (SAM).

The adsorption process for moderate concentrations (1 mM) of alkanethiol is characterized by a rapid initial phase during which the alkanethiol thickness rises to 80-90% of its maximum within a few minutes. This initial phase is followed by a slower period, lasting several hours, during which the alkanethiolate layer achieves its final thickness. It has been reported in the art that monolayers of alkanethiols on gold appear to be stable indefinitely in air or in contact with liquid water or ethanol at room temperature. The alkanethiolate-coated microparticles were recovered by centrifugation, washed twice in absolute ethanol and twice in triple-distilled water and stored at 4° C. in 1 ml of triple-distilled water.

The phospholipid layer was formed over the alkanethiol layer by combining alkanethiolate-coated microparticles with aqueous suspensions of DMPC small unilamellar vesicles. The DMPC vesicles were made by placing 1 ml of a 20 mg/ml DMPC in chloroform lipid solution into a round bottom flask and evaporating the solvent for several hours using a vacuum rotary evaporator. The dried lipid was resuspended in 50 μl of isopropanol and injected into 10 ml triple-distilled water while vortexing to form a suspension of large multilamellar vesicles. The solution of large vesicles was sonicated in a bath sonicator for several minutes to disrupt the large vesicles into small 20-100 nm unilamellar vesicles. This 10 ml suspension of small vesicles was combined with 10 mg of alkanethiolate-coated beads and gently mixed for 30 minutes at room temperature. The alkanethiolate-phospholipid-coated microparticles were recovered by centrifugation, washed twice in triple-distilled water and stored in triple-distilled water.

### Example 11

### Engineered Microparticle Testing

The analysis of the single-shell dielectric model predicts a definite relationship between the thickness of the outer insulating shell and the dielectrophoretic properties of an engineered dielectric microparticle. Engineered microparticles of appropriate thin-insulating-shell-over-conductive-interior composition are predicted to experience strong negative dielectrophoresis at frequencies between 10²-10⁹ Hz. In this frequency range, the electrical field would be unable to penetrate the outer insulating shell—from a dielectric perspective, the microparticle would have a high AC impedance and appear relatively non-polarizable. At frequencies in the 10⁷-10⁹ Hz range, engineered microparticles are predicted to experience strong positive dielectrophoresis. In this frequency range, the electrical field would penetrate the thin outer insulating shell via capacitive coupling and the core properties would dominate—dielectrically, the microparticle would have a low AC impedance and appear highly polarizable.

In the transitional 10⁵-10⁶ Hz range, increasing frequency is predicted to correlate with a change in the dielectrophoretic force acting on the engineered microparticle from decreasing negative to increasing positive. At the crossover frequency, f_{c}, the net dielectrophoretic force acting on the microparticle is zero—at frequencies below f_{c}, the particle experiences negative dielectrophoresis, and at frequencies above f_{c}, the particle experiences positive dielectrophoresis. An increase in the thickness of the insulating outer shell correlates with an increase in the crossover frequency. The relationship between the thickness of the insulating shell and the crossover frequency is given by the following relationships

$${C}_{mem} \approx \frac{{σ}_{s}}{R π \sqrt{2 {f}_{c}}}$$

$$and$$

$${C}_{mem} = \frac{{ϵ}_{0} {ϵ}_{mem}}{d}$$

where Cₘₑₘ is the specific membrane capacitance (i.e., normalized to the shell area), σₛ is the conductivity of the suspending medium, R is the radius of the engineered microparticle, f_{c} is the crossover frequency, ε₀ is the permittivity of free space, εₘₑₘ is the permittivity of the insulating layer, and d is the thickness of the insulating layer. Combining these equations yields the following relationship:

$$d ∝ \frac{{f}_{c}}{{σ}_{s}}$$

According to the equation above, the inverse slope of a plot of f_{c} versus σₛ gives the approximate specific membrane capacitance for a given engineered microparticle type. Furthermore, the slope of such a plot should increase with increasing membrane thickness.

Dielectrophoretic crossover frequency studies were performed by the inventors in order to determine whether four types of engineered dielectric microparticles (outer self assembled insulating monolayer of C₉ or C₁₈ alkanethiol or C₉ or C₈ alkanethiol +C₁₄ phospholipid) exhibited the predicted correlation between insulating shell thickness and crossover frequency. For the dielectrophoretic studies, the engineered dielectric microparticles were suspended in a DEP buffer containing 8.5% (w/v) sucrose, 0.3% (w/v) dextrose. The electrical conductivity of the buffer was adjusted with 300 mM EDTA (adjusted to pH 7.0 with NaOH). Aliquots of the engineered bead suspension were placed in an open reservoir above parallel electrode (50 μm trace-50 μm gap) of gold-on-glass construction that was energized with 1-10 volts peak-to-peak at frequencies between 1 kHz and 100 kHz to generate inhomogeneous electric fields for dielectrophoretic manipulation. Dielectrophoretic manipulation was accomplished by switching the field frequency, and the dielectrophoretic crossover frequency was determined.

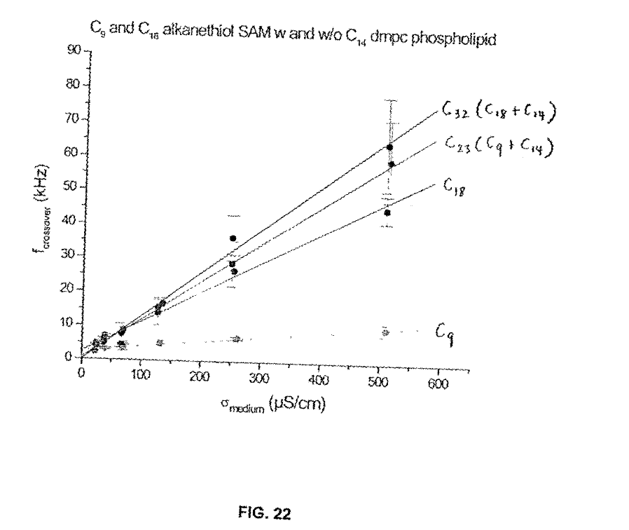

As shown by FIG. 22, the engineered microparticles show a definite dependence on thickness of the insulating outer shell as mediated by the choice of an alkanethiol and phospholipid of appropriate carbon chain length. Furthermore, the y-intercept occurs at very near zero, indicating the conductivity of the insulating layer is low and the self assembled monolayers provide a robust, uniform insulating layer.

Lipophilic molecules such as the pore-forming protein mellitin may be incorporated into the insulating layer. In addition, one may adsorb probes such as thiolated nucleic acids and proteins to the gold surface of the engineered microparticle core, and oligonucleotide and protein capture probes may be linked to the alkanethiolate and phospholipid molecules.

### Example 12

### Utilizing Dielectrically-Dispersive Materials

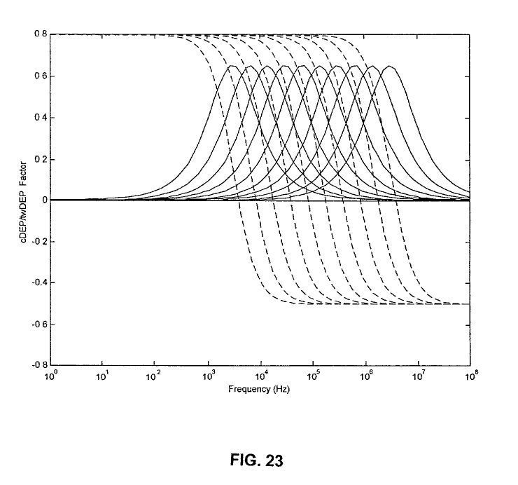

Many materials exhibit a high dielectric permittivity in low frequency AC electrical fields and a much lower permittivity at sufficiently high AC frequencies. In an intermediate, so-called dispersive, frequency range, the permittivity falls with increasing frequency (see FIG. 23). In FIG. 23, the solid lines show dielectric loss that gives rise to traveling wave dielectrophoresis and electrorotation while the dashed lines show dielectric permittivity (dielectric constant) that gives rise to conventional dielectrophoresis.

Without being bound by theory, the underlying physical mechanism for this frequency-responsive permittivity may include dipole alignment and space charge polarization at interfaces (so-called Maxwell-Wagner polarization) effects. The frequency dependence of the dielectric response is governed mostly by physical properties of the material that govern the rate at which charges rearrange in response to a changing electrical field.

In the case of dipolar materials, agents that act to increase the rate with which dipolar molecules can respond to a field increase the frequency at which the permittivity begins to fall. Conversely, agents that slow the response lower this frequency.

In one embodiment, chemical modification of dipolar materials may be used to bring about a change in the frequency response of the permittivity and hence can be used in a host of applications described herein and elsewhere. For example, the rate with which a dipolar long-chain molecule responds to a changing electrical field may be altered if the chain length of the molecule is modified. The response rate may be increased by decreasing the chain length and vice-versa. For instance, a dipolar molecule of chain length 9 will respond more quickly than a molecule of chain length 10, for example. Accordingly, one may tailor dielectric properties through such modifications. The tailored dielectric properties may be exploited as described herein for diverse applications such as the manipulation, indexing, isolation, separation, purification and identification of materials.

In one embodiment, a library of microparticles may be created having different “classes” of microparticle. Within each class of microparticle, the chain length of a particular molecule of the microparticle may be a certain length. In different classes, however, that molecule may be of a different length. Consequently, each class will possess distinct dielectric properties, which may be exploited as described herein for the manipulation, indexing, isolation, separation, purification and identification of materials. Different classes of the library may be independently addressed, manipulated, and characterized even when part of a mixture of multiple types of engineered microparticles. Depending upon the circumstances, individual microparticles within a particular class may also be independently addressed, manipulated, and characterized as dictated by the application. This ability to distinguish between classes and individual microparticles according to dielectric properties affords the practitioner with a flexible tool for many types of analysis, as will be recognized by those having skill in the art with the benefit of this disclosure.

The addition of side chains that modify steric interactions between dipolar molecules also alters the rate of response of dipolar materials to changing electrical fields; hence, adding (or removing or modifying) side chains may be used in applications described herein and elsewhere. For example, the replacement in a dipolar molecule of a methyl side chain by an ethyl side chain alters its dielectric response frequency. Similarly, replacement of a lysine side chain with an amine side chain alters the frequency response of a dipolar molecule. Similar modifications made to molecules within the dielectric material that are non-polar in nature may also be used to modify the frequency response of neighboring dipoles.

In one embodiment, a library of microparticles may be created having different classes of microparticle based upon the addition of different (or the addition of no) side chains. Each class will possess distinct dielectric properties, which may be exploited as described herein for the manipulation, isolation, separation, purification and identification of materials.

The dielectric response of a material may also be modified by altering the chemical composition of the material and/or the physical processes used to manufacture or subsequently treat the material. For example, the dielectric, ferroelectric, and antiferroelectric properties of PbTiO₃, Pb(ZrxTi1−x)O₃, BaTiO₃, SrTiO₃, KTaO₃, and many other materials may be greatly affected by changes in chemical composition by doping with agents such as, but not limited to, Ba, and by thermal treatment following formulation to alter the domain and matrix structure. Accordingly, by controlling such parameters, one may obtain materials with modified, distinct dielectric properties. By altering the properties in one microparticle in a manner different than for another microparticle, one may build a dielectric library of microparticles, each particle (or class of particles) having a different, distinct dielectric profile. The differences in those dielectric profiles may then be exploited for, for example, the separation or general analysis of analytes in microfluidic or other applications.

As will be understood by those having skill in the art, a wide array of other doping agents and modifications to manufacturing that affect dielectric responses may be employed to arrive at one or more apparatuses or methods described and claimed herein.

In the case of space-charge polarization, agents that increase the mobility of charge carriers increase the frequency at which the permittivity starts to fall, while agents that decrease the mobility have the opposite effect. For example, a lipid vesicle may be filled with an ionic solution in order to produce a dielectric microparticle. Modification of the viscosity of the medium within the vesicle by addition of agents such as PEG, agarose, and other viscous agents known in the art, reduces the mobility of charge carriers within the vesicle and thereby reduces the frequency at which the permittivity begins to fall. Using such techniques, one is afforded yet another way to create materials having a readily definable dielectric response characteristic; these materials, in turn, can be utilized in applications such as those described herein.

Similarly, charge carriers having different mobilities within the same medium may be used to produce the same or a similar effect. As an example, a solution of (NH₄)₂SO₄ contains bulkier and less mobile charge carriers than a solution of NaCl, for which the charge carriers are smaller and more mobile. This solution, or others having similar properties may correspondingly be used as the basis of a microparticle having a distinct, engineered dielectric response.

It follows from the above non-limiting exposition that by generally altering the composition or makeup of a dielectric material and/or the conditions under which it is formulated and subsequently treated, one may readily influence the frequency response characteristics of the dielectric. FIG. 23 shows the predicted response of a family of dielectric particle types that are of substantially the same composition and structure but that contain different amounts of an agent that alters the rate of charge reorganization in response to an electrical field. As is illustrated, the small variations in composition lead to desirable differences in the dielectric properties and electrokinetic responses that make them applicable for use as dielectric labels, indexes or carriers.

With the benefit of this disclosure, those having skill in the art will recognize that by altering the composition or makeup and/or conditions of manufacture as described in this example, one may make a dielectric material having desirable response characteristics tailored for one or more different applications. For instance, one may use any one of the techniques of this example, alone or in combination, in a controlled, methodical way (for instance, by adding one or more chemical agents or by changing the way raw materials are heat processed) to manufacture a library of beads. Within such a library, each bead may be made to have a slightly different, but distinctly discernable, dielectric response. Those beads may then be used, for example, upon a reaction surface utilizing dielectrophoretic manipulation forces to carry out a myriad of microfluidic studies or applications.

The embodiments of this example are but examples of the range of modifications that may be employed for the purpose of altering the dielectric response of a material. Using the teachings herein, one of skill in the art may perform similar modifications to adjust the dielectric properties of microparticles used as, for instance, labels, indexes, or carriers.

It will also be understood that materials such as those described in this example may be used to fabricate entire engineered microparticles, to form one or more cores or layers of an engineered microparticle, or any other portion of an object useful for microfluidic or other applications.

### Example 13

### Utilizing Surface Charge

In addition to the techniques of Example 12, one may alter the electrokinetic responses of a material by modifying the electrostatic interaction of its surface with a suspending medium. For example, if a particle's surface is charged, it will induce in the suspending medium a so-called charge double layer comprised of a cloud of counter ions.

One may create a family or class of microparticles identical except for surface charge properties. When placed in an electrical field, the charge double layer acts as an additional dielectric layer with its own polarizability and frequency response characteristics. In a non-limiting example, this additional dielectric layer may be engineered via intentional changes to surface charge properties so that a plurality of microparticles may be manufactured, each microparticle (or class of microparticle) having a different dielectric response characteristic. Such microparticles may then be utilized in applications such as those described herein and elsewhere. For instance, they may be used on a reaction surface that uses dielectrophoretic manipulation forces to direct one or more microfluidic processes.

The electrokinetic properties of particles are dependent on the charge double layer characteristics; hence, electrokinetic properties including but not limited to DEP collection and DEP-FFF properties may be engineered by chemically and/or physically modifying the surface charge. Charge double layer effects are pronounced at frequencies below 1 kHz for particles of 10 micron diameter in an aqueous medium having a conductivity of ˜20 mS/m. The frequency of the dielectric dispersion of the charge double layer increases with decreasing particle size and with increasing suspension conductivity.

Methods to alter the surface charge include but are not limited to: addition of carboxy, amino, or other charged groups, and removal of charge by neuraminidase or other enzymic or chemical treatments known in the art. These treatments may be applied to any material useful in microfluidic applications and other applications, including general microparticles used as, for instance, labels, indexes, or carriers., beads, and the like.

As will be understood by those having skill in the art with the benefit of this disclosure, the engineering of surface charge as described in this example may be applied in a wide range of additional applications, including but not limited to applications that facilitate the use of electrophoresis in combination with electrokinetic methods.

### Example 14

### Utilizing Fluorescent Labeling

It is known that fluorescence-based methods may be used for indexing microparticles for the purpose of discriminating between multiple analytes in a mixture through the use of multiple dyes. In response to some excitation radiation, each dye possesses a discernibly different fluorescent response, which acts to distinguish different microparticles (and/or analytes). While this method facilitates the identification of different particles in a mixture, it does not provide a means for manipulating them. Furthermore, the number of different particle types that can be discriminated by the conventional methodology is limited.

These limitations may be overcome, however, by adding dielectric discrimination and manipulation capabilities to microparticles labeled according to fluorescence-based methods. In this way, microparticles may be trapped, focused, fractionated, isolated and otherwise manipulated by electrokinetic methods as described herein. Furthermore, by introducing dielectric discrimination as a parameter of the microparticle, an independent, additional discriminating parameter is provided. This addition greatly increases the total number of microparticle types that can be discriminated in an experiment. With this additional parameter, for example, larger and more precise libraries of beads or other particles may be manufactured that can be used in a vast array of applications described herein and elsewhere.

As will be understood by those having skill in the art, the dielectric discrimination parameter may be added to fluorescently-labeled microparticles by using any one (or combination) of methods taught herein.

### Example 15

### Utilizing the Combination of Different Particle Types

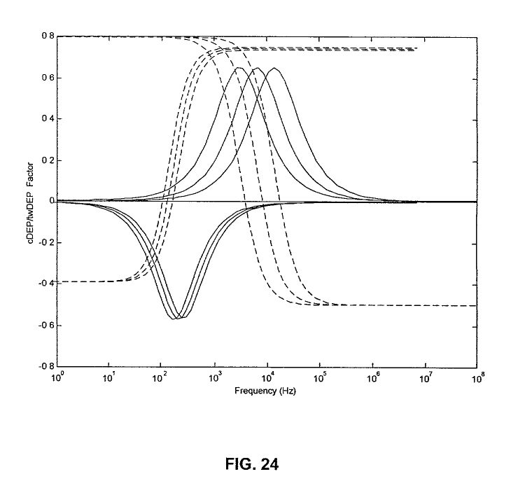

One may use mixtures of microparticles that have been engineered in different ways in order to produce desirable dielectric properties. For example, FIG. 24 illustrates the frequency responses of two types of microparticles: three engineered through a self- assembled, insulator-over-conductive-core, biomimetic approach, and three through a dielectric-dispersive-core approach. As illustrated, the biomimetic microparticles exhibit a low permittivity at low frequencies that increases with increasing frequency. Conversely, the dispersive-core microparticles exhibit a high permittivity at low frequencies that decreases with increasing frequency.

Thus, these two particle families have distinctively different frequency-dependent dielectric properties that may be exploited in order to manipulate them differentially by electrokinetic methods or to identify them by impedance sensing methods such as but not limited to those disclosed in pending U.S. application Ser. No. ______ entitled, “Particle Impedance Sensor” by Gascoyne et al., filed Dec. 3, 2001, which is hereby incorporated by reference.

With the benefit of this disclosure, it will be understood that any combination of different particle types taught herein may be similarly exploited to result in the obtainment of particles having distinct electrical properties that can then be used for (for example) separation, manipulation, purification, and indexing of analytes. Similarly, any combination of techniques taught herein may be combined in the production of a single microparticle having a distinct, engineered dielectric response.

### Example 16

### Microparticle Aggregation

Microparticles with little or no surface charge may tend to associate in aqueous suspension media, forming aggregates. Depending upon the application, aggregation may or may not be desirable, and its degree of aggregation may similarly be dependent upon the application.

Aggregation tendencies may be enhanced or diminished by modulating the surface charge on the surface of a microparticle. In cases where aggregation is undesirable, the surface of a microparticle may be modified to increase the net surface charge. For example, the inventors have used chemically bound carboxyl and amino groups, as well as physically bound long chain fatty acids and gangliosides to increase the net surface charge of microparticles. As a result, the aggregation of those microparticles was greatly inhibited.

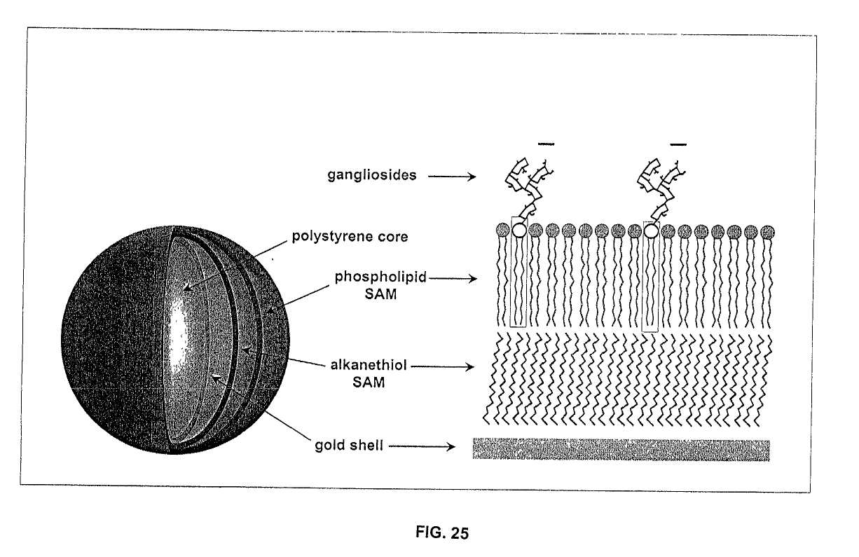

Turning to FIG. 25, there is shown a schematic diagram of one embodiment of an engineered microparticle incorporating gangliosides to modify the net surface charge of the microparticle and, hence, to control its aggregation. FIG. 25 shows both a view of the microparticle and a view representing the layers thereupon. As illustrated, the exemplary microparticle includes a polystyrene core, a gold shell, an alkanethiol SAM, a phospholipid SAM, and gangliosides. As can be seen by reference to the view showing each layer, the gangliosides, which result in a negative charge, may be incorporated generally with the phospholipid SAM layer. As will be understood by those having skill in the art, the particular depiction of the microparticle of FIG. 25 is exemplary only, and other types of microparticles as taught herein may be substituted for that of this figure.

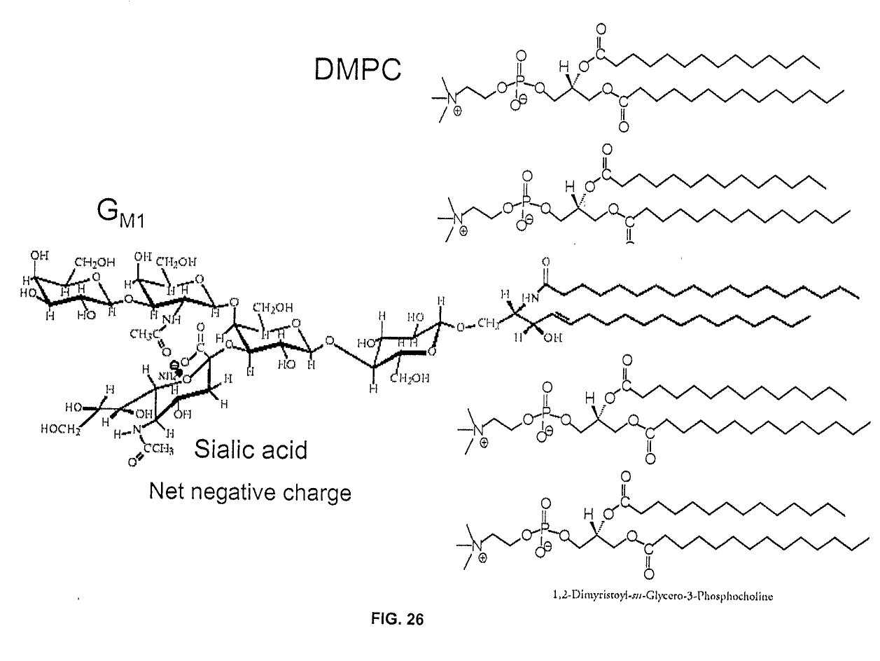

FIG. 26 is a more chemically-detailed schematic diagram showing how, in one embodiment, gangliosides may be incorporated with microparticles. Here, there is shown a DMPC layer, which may correspond to the phospholipid SAM layer of FIG. 25. Incorporated with the DMPC layer exists the illustrated GM1 ganglioside, including the illustrated sialic acid. In one embodiment, a ratio of DMPC to gangliosides may be about 20:1, although it will be understood that any other ratio suitable for affecting surface charge may also be used. It will also be understood that any ratio may be modified within different classes of microparticles to create a library of microparticles. In other words, one class of microparticles may exhibit a 20:1 ratio, while a different class of particles may exhibit a ratio of about 35:1.

As noted in the figure itself, the embodiment of FIG. 26 provides a net negative charge, which as described above, influences aggregation; in particular, this arrangement allows one to reduce or eliminate “sticking” between and among microparticles. By modifying the surface charge of some microparticles more or less than others, one may create a spectrum of different aggregation properties that may be exploited, depending upon the particular application. This spectrum may serve as the basis for the creation of libraries of microparticles.

In other embodiments, one may use different gangliosides, including but not limited to GD1a gangliosides with two sialic acid residues/molecules. In other embodiments, one may monitor the incorporation of the gangliosides by, for instance, labeling with FITC-conjugated cholera toxin B or another appropriate material. One may utilize any one of various scattering experiments to quantitate aggregation, and one may use such quantitation as a feedback mechanism to perfect the engineering of a particular library of microparticles so that their individual or composite aggregation characteristics are ensured to be as-desired.

With the benefit of the present disclosure, those having skill in the art will understand that a number of different surface modification techniques can be used to modify surface charge, which in turn modifies aggregation properties.

### Example 17

### General Vesicles

In addition to the techniques described above, one may engineer dielectric response characteristics by building customized vesicles having specific dielectric properties. Different customized vesicles may be made with different properties so that that microparticle libraries may be formed.

In one embodiment, one may produce unilamellar or multilamellar vesicles by appropriately mixing lipid or polymer materials with polar media. The resulting vesicles encapsulate the medium in which they were prepared. By this means, one may engineer microparticles having desired dielectric properties for use in indexing, labeling or carrier applications.

In one embodiment, different dielectric properties may arise due to differences in the contents of different vesicles. For example, in one class of microparticles, each microparticle may encapsulate the same medium. In another class, each vesicle may encapsulate a similar, albeit sufficiently different medium to give rise to a discernible different in dielectric response. In another embodiment, the vesicle itself may be modified in a controllable manner so that different types of vesicles exhibit a different dielectric response, even if the contents of different vesicles are identical.

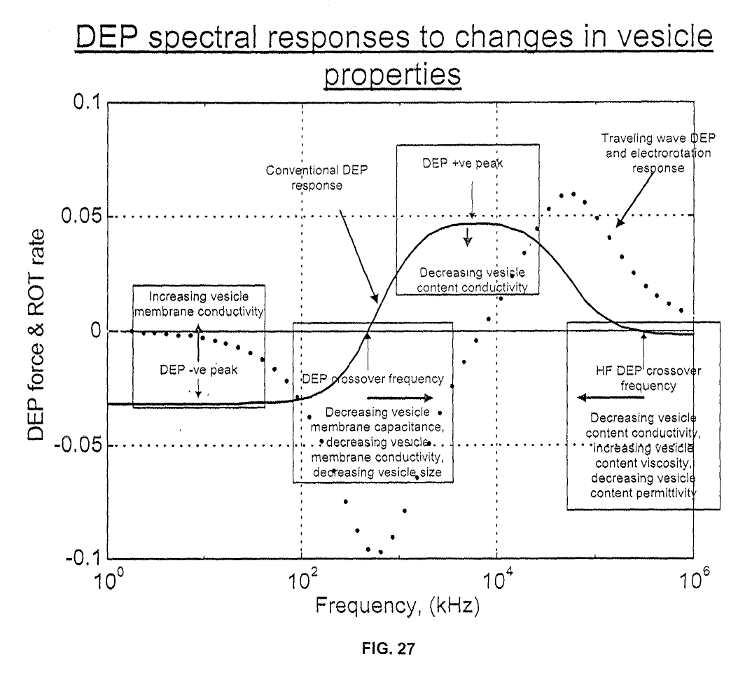

Turning to FIG. 27, there is shown a graph with accompanying comments concerning dielectrophoretic spectral responses to changes in vesicle properties. This graph illustrates how a vesicle's size, membrane capacitance, membrane conductivity, content permittivity, content conductivity, and content viscosity can all act as parameters that can be modified in order to obtain a particular dielectric response characteristic. In other words, any one or combination of these (or similar) parameters may be adjusted until one obtains a microparticle of desired characteristics. Different classes of microparticles may be adjusted differently so that a library of microparticles may be created. In this regard, FIG. 27 is instructive in that it shows general trends associated with the different parameters; by following these trends set forth on the graph, one may readily create a library of vesicle-based microparticles without any undue experimentation.

### Example 18

### Erythrocyte Ghosts

As is known in the art, one may suspend cells such as erythrocytes in hypotonic media and bring about their rupture so that their contents are lost. Cells that have undergone this processing are sometimes called “ghosts.” As is also known in the art, by appropriately suspending ghosts in a modified medium, one may induce them to reseal. The ghosts thereby encapsulate their suspending medium. This example teaches how one may use ghosts to form dielectric microparticles having engineered dielectric characteristics.

In one embodiment, ghosts can be made using the following instructions: (a) wash normal erythrocytes 3 times in PBS, (b) lyse erythrocytes in 5 mM TRIS+0.25 mM EDTA plus 0.12 mM PMSF, (c) spin down at 15,000 RPM, (d) do two more washes and spins, and (e) reseal ghosts at 37C., 30 minutes in the medium to be encapsulated. In some embodiments, one may contact ghosts with percoll (for density) and high MW salts (for interior conductivity without ion-channel permeability). Other methodology known in the art may also be used to create ghosts.

By appropriate choice of conditions, the dielectric properties of the resealed ghosts may be adjusted to produce microparticles with desirable properties for use in indexing, labeling or carrier applications. For example, in one embodiment, one may produce different ghosts, each one having different dielectric properties arising from, for example, their contents and/or from inherent dielectric properties contributable to the (empty) ghosts themselves.

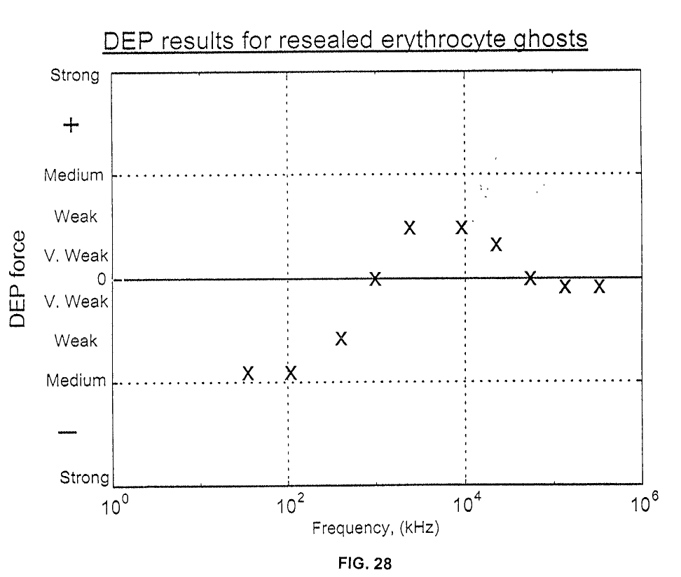

FIG. 28 shows DEP results for resealed erythrocyte ghosts according to one embodiment of the present disclosure.

### Example 19

### Specific Engineered Microparticle Library

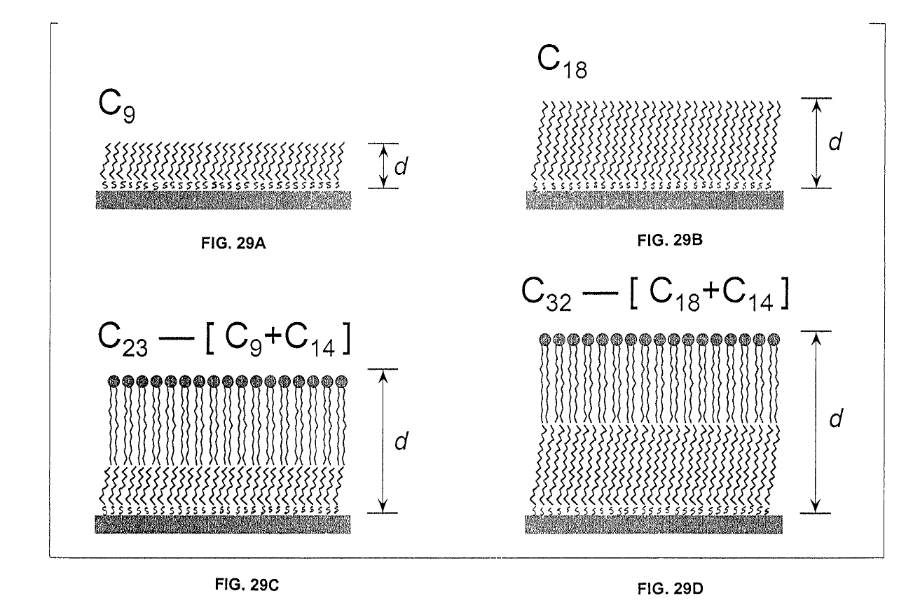

Turning to FIGS. 29A -29D, there is shown one embodiment of a series of specific microparticles (the views showing layers thereof), which may be included as part of an engineered microparticle library as described herein. Shown are microparticles having a C9 layer, a C18 layer, a C23 layer (made up of C9 and C14 layers), and a C32 layer (made up of C18 and 14 layers). As can be seen the effective diameter of the microparticles may vary widely depending upon the length of the chain molecules making up one or more layers. This, in turn, affects the dielectric response characteristics as described herein.

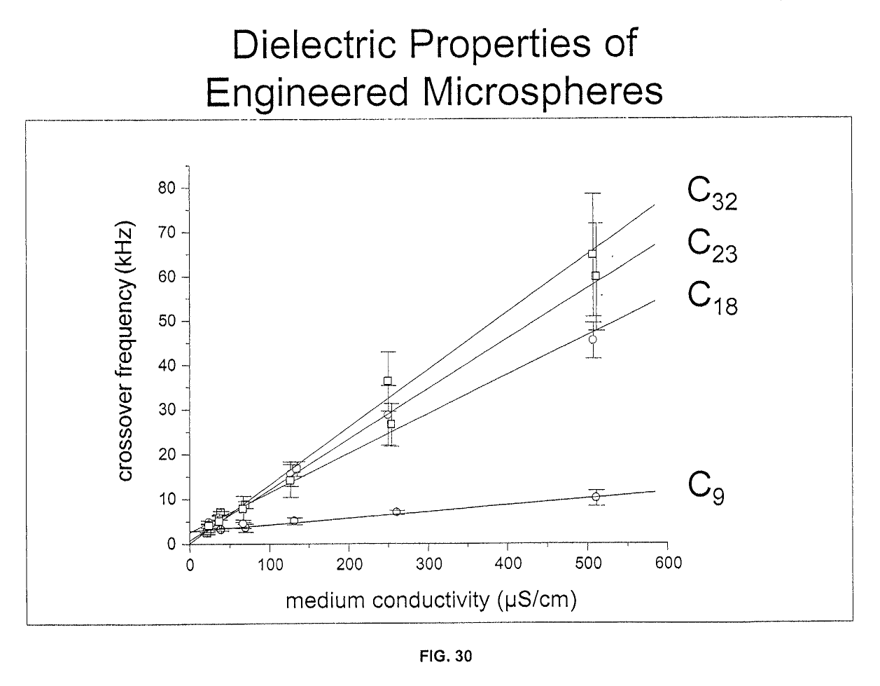

Representative data for the microparticles of FIG. 29A-29D is depicted in FIG. 30.

### Example 20

### Biotin/Streptavidin System for Surface Functionalization

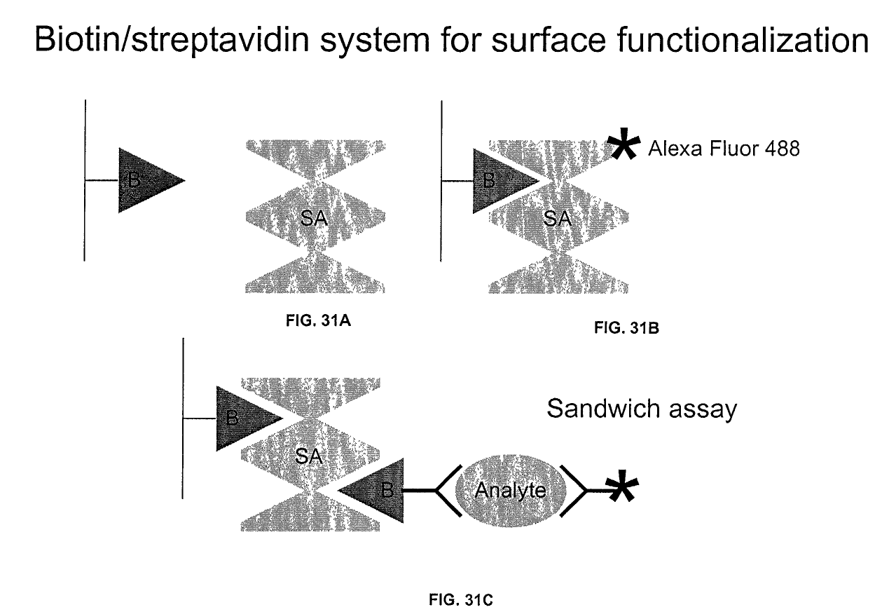

FIGS. 31A-31C illustrate a system according to embodiments of the present disclosure in which a biotin/streptavidin system may be employed for surface functionalization for use with the apparatuses and methods described herein. Biotin is represented by the “B,” and streptavidin by the “SA.” As illustrated, the biotin/streptavidin system may be employed as a general mechanism about surfaces of microparticles so that one may add different functional mechanisms to the surfaces of microparticles to accomplish, for example, life-science applications. Shown in FIG. 31A-31C are applications in which a sandwich assay is created using the biotin/streptavidin system.

Advantages of using such a system include but are not limited to: biotin-streptavidin binding is very tight, biotinylated antibodies are readily available, and both proteins and oligonucleotides can be readily biotinylated.

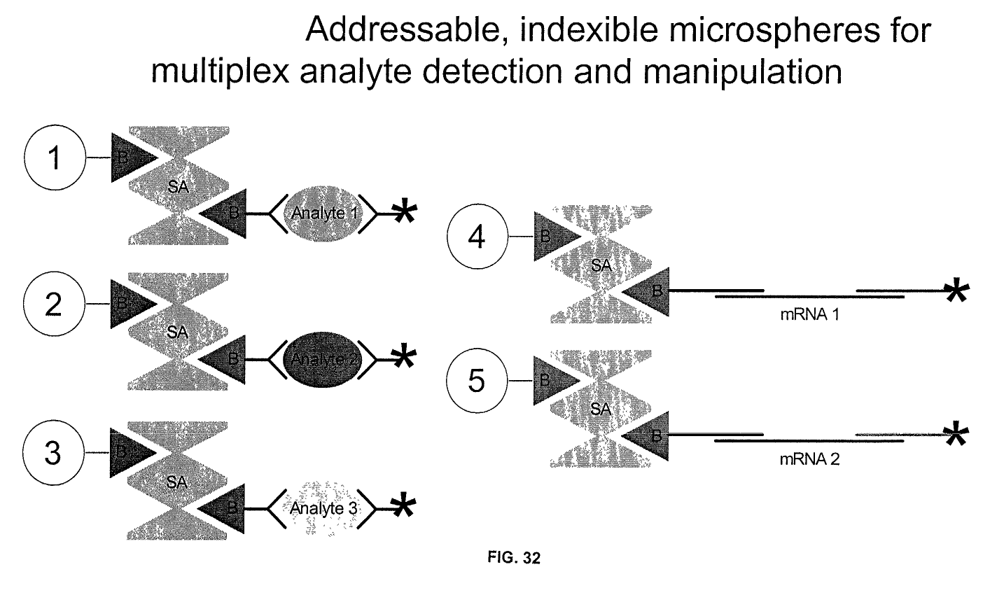

FIG. 32 is a schematic diagram according to embodiments of the present disclosure showing a biotin/streptavidin system for addressable, indexible microparticles for multiplex analyte detection and manipulation. In particular, the B/SA system may be employed with a library of microparticles. One may probe a sample for multiple protein/mRNA samples simultaneously, and microparticle beads may be identified and/or manipulated as taught herein. In one embodiment, different beads may be identified via impedance sensing. In another embodiment, beads may be identified by fluorescence data. In yet another embodiment, a combination of position and fluorescence data may be utilized for identification.

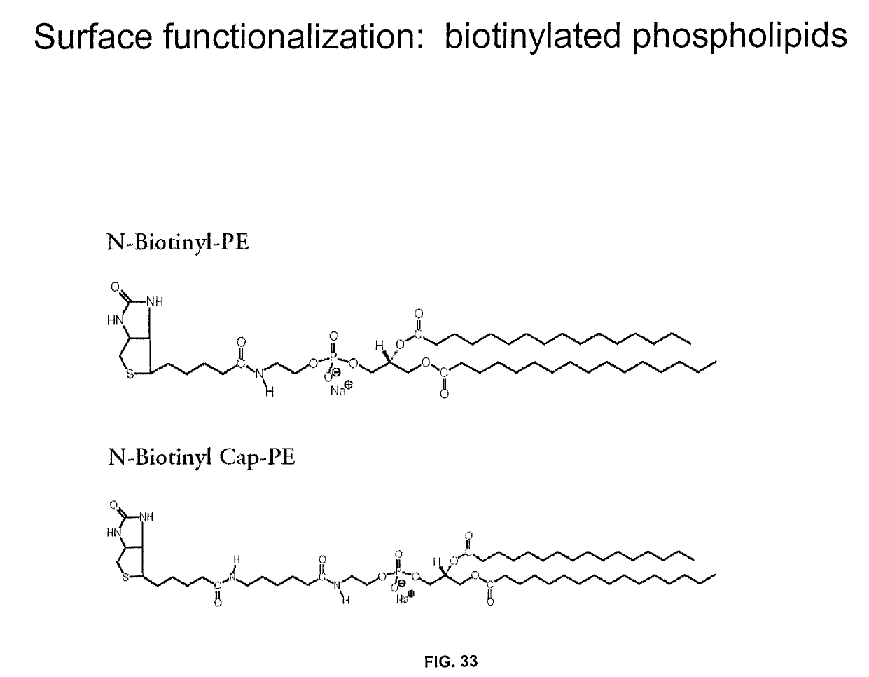

FIG. 33 is another schematic diagram according to embodiments of the present disclosure, showing a more-detailed chemical illustration of surface functionalization by biotinylated phospholipids. This surface functionalization may be employed with apparatuses of this disclosure by attaching to one or more lipid layers of an engineered microparticle. This may provide for a universal surface attachment method, offering great flexibility in terms of applications that can be performed.

In one embodiment, biotinylated lipids may be incorporated into one or more DMPC/ganglioside layers, although incorporation may be done into different layers as will be understood by those having skill in the art. In another embodiment, one may covalently link molecular species for functionalizing surfaces using the Diels-Alder reaction.

In different embodiments of the present disclosure, a mixture of DEMPs having different dielectric properties and different surface functionalizations may be used as handles to facilitate the separation of one or more classes of substances from a mixture so that these separate classes may be subjected to additional analyses tailored to each individual class.

For example, a first set of DEMPs may be functionalized to target protein molecules of one or more kinds. Another set may be functionalized to target nucleic acids. Still another may be functionalized to target classes of lipids. Still another might be functionalized to target steroids. A mixture of these differently functionalized sets of DEMPs may be combined with a suspension of, for example, mammalian cells that were then burst, releasing the molecular contents of the cells. Each set of DEMPS bind the molecular types for which it had been functionalized.

Subsequently, dielectric or combined dielectric and magnetic methods may be used to sort the different DEMP sets into different reaction spaces, where separate analysis protocols may be applied to analyze each class of target molecules. For example, proteins may be analyzed by protein chemistries, nucleic acids by nucleic acid protocols, and so on. Those skilled in the art having the benefit of this disclosure will recognize that this methodology may be extended to provide for the analysis of many different classes of analytes from mixtures. For example, different types of organelles, cells, bacteria, viruses, prions, pollens, spores, or other types of biological entities, and different types of silts, sediments, soils, aerosols, smokes, and other inert particles may be separated for additional processing using this approach.

One non-limiting advantage of the DEMP method for collecting target molecules is that the DEMPs may be collected or trapped by positive DEP, while reagents are flowed over them. In this way, target molecules may be exposed to desired reagents following their capture and unwanted residual materials, such as cell debris or unwanted molecules with weak affinity to the DEMPs, may be washed away or otherwise depleted from the DEMPs to leave the target species in a purer form.

All of the compositions and/or methods disclosed and claimed herein can be made and executed without undue experimentation in light of the present disclosure. While the compositions and methods of this invention have been described in terms of specific embodiments, it will be apparent to those of skill in the art that variations may be applied to the compositions and/or methods and in the steps or in the sequence of steps of the method described herein without departing from the concept, spirit and scope of the invention. For instance, as will be understood with reference to this disclosure, dielectric or conductive properties, especially of semiconductive particles, shells or cores, may be affected by heat and/or by light, allowing yet another level of control or discrimination (in addition to those disclosed above). Additionally, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Abidor, Li, Sowers, “Membrane electrofusion yeilds in membrane fractions obtained with a colloid-osmotic hemolysis and electrohemolysis procedure,” Bioelectrochem. Bioenerget. 34, 31-37, 1994.

Arnold, Zimmermann, “Electro-rotation: developments of a technique for dielectric measurements on individual cells and particles,” J. Electrost. 21 151-191, 1988.

Arnold, Zimmermann, “Rotation of an isolated cell in a rotating electric field,” Naturwissenschaften 69 297-300, 1982.

Barany, Proc. Natl. Acad. Sci. USA 88:189, 1991.

Dutton et al., Proc. Natl. Acad. Sci., 76, 3392-3396, 1979.

Elshabini, Barlow, in Thin Film Technology Handbook, McGraw-Hill, New York, Ch 1, 1998.

Fuhr, “Über die rotation dielektrischer korper in rotierenden feldern,” PhD Dissertation Humboldt-Universität, Berlin Chap. 3, pp24-53, 1985.

Fuhr, Rosch, Müller, Dressler, Goring, “Dielectric spectroscopy of chloroplasts isolated from higher plants—characterization of the double-membrane system,” Plant Cell Physiol. 31, 975-985, 1990.

Gascoyne, Huang, Hughes, Wang, Pethig, Becker, “Manipulations of biological cells using travelling-wave dielectrophoresis,” Proc. 16th IEEE:Eng. Med. Biol. Soc. 772-773, 1994a.

Gascoyne, Pethig, Burt, Becker, “Membrane changes accompanying the induced differentiation of friend murine erythroleukemia cells studied by dielectrophoresis,” Biochim. Biophys. Acta 1149, 119-126, 1993.

Gascoyne, Wang, Becker, “Numerical analysis of the influence of experimental conditions on the accuracy of dielectric parameters derived from electrorotation measurements,” Bioelectrochem. Bioenerg. 36, 115-125, 1994b.

Gefter et al., Somatic Cell Genet., 3: 231-236, 1977.

Gellert, Küstner, Hellwich, Kastner, in The VNR Concise Encyclopedia of Mathematics (Van Nostrand Reinhold, New York) pp 420-424, 1977.

Geohegan et al., Immunol. Comm 7:1, 1978.

Gimsa, Marszalek, Loewe, Tsong, “Dielectrophoresis and electrorotation of neurospora slime and murine myeloma cells,” Biophys. J. 60, 749-760, 1991.

Goding, 1986, In Monoclonal Antibodies: Principles and Practice, 2d ed., Orlando, Fla., Academic Press, 1986, pp. 60-61, 65-66, and 71-74.

Guatelli et al., Proc. Natl. Acad. Sci. USA 87:1874-1878, 1990.

Hagedorn, Fuhr, Müller, Gimsa, “Travelling-wave dielectrophoresis of microparticles,” Electrophoresis 13:49-54, 1992.

Hölzel, Lamprecht, “Dielectric properties of yeast cells as determined by electrorotation,” Biochim. Biophys. Acta 1104,195-200, 1992.

Hölzel, Lamprecht, Mischel, “Characterisation of cells by dielectrophoretic techniques,” in Physical Characterization of Biological Cells, eds. Schütt, W., Klinkmann, H., Lamprecht, I. & Wilson, T. (Verlag Gesundheit Gmh, Berlin), 1991.

Hu, Arnold, Zimmermann, “Alterations in the electrical properties of T and B lymphocyte membranes induced by mitogenic stimulation. Activation monitored by electro-rotation of single cells,” Biochim. Biophys. Acta 1021, 191-200, 1990.

Huang, Holzel, Pethig, Wang, “Differences in the AC electrodynamics of viable and nonviable yeast cells determined through combined dielectrophoresis and electrorotation studies,” Phys. Med. Biol. 37, 1499-1517, 1992.

Huang, Wang, Tame, Pethig “Electrokinetic behaviour of colloidal particles in travelling electric fields: studies using yeast cells,” J. Phys. D: Appl. Phys. 26:1528-1535, 1993

Irimajiri, Hanai, Inouye “A dielectric theory for “multi-stratified shell” model with its application to a lymphoma cell,” J. Theor. Biol. 78 251-269, 1979.

Ithakissios et al., Clin. Chem., 23, 2072-2079, 1977.

Jones, Electromechanics of Particles, Cambridge University Press, Cambridge, 1995.

Jones, Kallio, “Dielectrophoretic levitation of spheres and shells,” J. Electrostat. 6 207-224, 1979.

Kaler and Jones, “Dielectrophoretic spectra of single cells determined by feedback-controlled levitation,” Biophys. J. 57:173-182, 1990.

Kohler and Milstein, Eur. J. Immunol., 6:511-519, 1976.

Kohler and Milstein, Nature, 256:495-497, 1975.

Landergren et al., Science 241:1077, 1988.

Lim, in Biomedical Applications of Microencapsulation, CRC Press, Boca Raton, 1984.

Lomeli et al., Clin. Chem. 35:1826, 1989.

Margessi et al., Clin. Chim. Acta., 89, 455-460, 1978.

Masud, Washizu, Kawabata, “Movement of blood-cells in liquids by nonuniform traveling fields,” IEEE Trans. Ind. Appl. 24:217-223, 1988.

Meuse, C. W., Fu, J., Conner, J. T., Plant, A. L. (1998). Construction of hybrid bilayers in air. Biophys. J. 74. 1388.)

Meuse, C. W.. Niaura, G., Lewis, M. L., Plant, A. L. (1998) Assessing the molecular structure of supported hybrid bilayer membranes with vibrational spectroscopies. Langmuir 14. 1604.

Milday et al., FEBS, 170, 232-238, 1984.

Molday et al., J. ImmunoL. Meth., 52, 353-367, 1982.

Molday et al., Nature, 268, 437, 1977.

Müller, Gerardino, Schnelle, Shirley, Bordoni, De Gasperis, Leoni, Fuhr, J. Phys D: Appl Phys 29:340, 1996.

Nye et al., Clin. Chim. Acta., 69, 387-396, 1976.

Oldenburg et al., “Nanoengineering of optical resonances,” Chemical Physics Letters 288 (1988) pp. 243-247 (May 1998).

Patterson et al., Science 260:976, 1993.

Pethig, Dielectric and Electronic Properties of Biological Materials, J. Wiley & Sons, New York, 1979.

Pethig, Markx, Trends in Biotech 15:426, 1997.

Plant, A. L. Self assembled phospholipid/alkanethiol biomimetic bilayers on gold. (1993) Langmuir 9: 2764-2767. [Online abstract]

Plant, A. L., Guegetchkeri, M., Yap, W. (1994) Supported phospholipid/alkanethiol biomimetic membranes: Insulating properties. Biophys. J. 67: 1126-1133. [Online abstract]

Plant, A. L, Gueguetchkeri, M. (1994) Planar phospholipid bilayer membranes formed form self-assembled alkanethiol monomers. Proceedings of the 13th Southern Biomedical Engineering Conference.

Plant, A. L., Brigham-Burke, M., O'Shannessy (1995) Phospholipid/alkanethiol bilayers for cell surface receptor studies by surface plasmon resonance. Anal. Biochem. 226:342-348. [Online abstract]

Pohl in Dielectrophoresis, Cambridge Universiy Press, Cambridge, 1978.

Rao, N. M., Plant, A. L., Hui, S. W., Silin, V., Wight, S. (1997) Characterization of biomimetic surfaces formed from cell membranes. Biophys. J. 73: 3066-3077. [Online abstract]

Rosenweig, Scien. Amer., 252, 10,136-194 (1983).

Saiki et al., Science 230:1350, 1985.

Saiki et al., Science 239:487, 1988.

Sauer, “Interaction-forces between microscopic particles in an external electromagnetic field,” in Interactions Between Electromagnetic Fields and Cells ed A Chiabrera et al (New York: Plenum) pp 181-202, 1985.

Sukhorukov, Arnold, Zimmermann, “Hypotonically induced changes in the plasma membrane of cultured mammalian cells,” J. Membr. Biol. 132, 27-40, 1993.

U.S. Pat. No. 5,993,630.

U.S. Pat. application Ser. No. 09/249,955.

Walker et al., Nucl. Acids Res. 20:1691, 1992.

Wang, Huang, Becker, Gascoyne, “A Unified theory of dielectrophoresis and travelling wave dielectrophoresis,” J. Phys. D: Appl. Phys. 27, 1571-1574, 1994a.

Wang, Huang, Burt, Markx, Pethig, “Selective dielectrophoretic confinement of bioparticles in potential energy wells,” J. Phys. D: Appl. Phys. 26, 1278-1285, 1993.

Wang, Huang, Gascoyne, Becker, Hölzel, Pethig, “Changes in Friend murine erythroleukaemia cell membranes during induced differentiation determined by electrorotation,” Biochim. Biophys. Acta 1193, 330-344, 1994c.

Wang, Hughes, Huang, Becker, Gascoyne, “Non-uniform spatial distributions of both the magnitude and phase of AC electric fields determine dielectrophoretic forces,” Biochim. Biophys. Acta. 1243:185-194, 1994b.

Wang, Vykoukal, Becker, Gascoyne “Separation of polystyrene microbeads using DEP/G-FFF,” Biophysics Journal 74:2689, 1998.

Widder et al., Clin. Immunol. and Immunopath., 14,395-400, 1979.

Wu et al., Proc. Natl. Acad. Sci. USA 89:11769, 1992,

Wu, Sjodin, Sowers, “Distinct mechanical relaxation components in pairs of erythrocyte ghosts undergoing fusion,” Biophys. J. 66, 114-119, 1994.

What is claimed is:

1. An engineered microparticle fabricated to be dielectrically-dispersive and adapted to produce a dielectric response to an applied electric field such that the microparticle is maneuverable by dielectrophoresis.

2. The microparticle of claim 1, comprising a dielectrically-dispersive core.

3. The microparticle of claim 2, comprising an insulating layer surrounding the core.

4. The microparticle of claim 3, wherein the insulating layer comprises a self-assembled monolayer.

5. The microparticle of claim 1, comprising streptavidin.

6. The microparticle of claim 5, comprising one or more biotinylated probes coupled to the streptavidin.

7. The microparticle of claim 1, comprising a dipolar material.

8. The microparticle of claim 1, comprising a doping agent.

9. The microparticle of claim 1, comprising a fluorescent label.

10. The microparticle of claim 1, comprising a ganglioside.

11. The microparticle of claim 1, comprising a vesicle.

12. The microparticle of claim 11, wherein the vesicle comprises an erythrocyte ghost.

13. A library of two or more engineered microparticles, each microparticle comprising a dielectrically-dispersive material differing to an extent sufficient to cause a discernible difference in a dielectric response of each microparticle to an applied electric field.

14. The library of claim 13, wherein the dielectrically-dispersive material comprises one or more layers of one or more materials.

15. The library of claim 13, wherein one or more of the engineered microparticles comprises a dielectrically-dispersive core.

16. The library of claim 13, wherein one or more of the engineered microparticles comprises a self-assembled monolayer.

17. The library of claim 13, wherein one or more of the engineered microparticles comprises streptavidin.

18. The library of claim 17, wherein one or more of the engineered microparticles comprises a biotinylated probe coupled to the streptavidin.

19. The library of claim 13, wherein one or more of the engineered microparticles comprises a fluorescent label.

20. The library of claim 13, wherein one or more of the engineered microparticles comprises a ganglioside.

21. The library of claim 13, wherein one or more of the engineered microparticles comprises a vesicle.

22. The library of claim 21, wherein the vesicle comprises an erythrocyte ghost.

23. The library of claim 21, wherein dielectrically-dispersive materials differ with respect to material encapsulated by two or more vesicles.

24. The library of claim 13, wherein dielectrically-dispersive materials differ with respect to chain length.

25. The library of claim 13, wherein dielectrically-dispersive materials differ with respect to a doping agent.

26. The library of claim 13, wherein dielectrically-dispersive materials differ with respect to methods of manufacture.

27. The library of claim 26, wherein dielectrically-dispersive materials differ with respect to heat treatments during manufacture.

28. The library of claim 13, wherein dielectrically-dispersive materials differ with respect to composition.

29. The library of claim 13, wherein dielectrically-dispersive materials differ with respect to surface charge.

30. The library of claim 13, wherein dielectrically-dispersive materials differ with respect to a side chain.

31. The library of claim 13, wherein dielectrically-dispersive materials differ with respect to mobility of charge carriers.

32. The library of claim 13, wherein dielectrically-dispersive materials differ with respect to viscosity.

33. A method for forming a library of two or more engineered microparticles, comprising:
obtaining a first dielectrically-dispersive material having a first dielectric property;
modifying the first dielectrically-dispersive material to form a second dielectrically-dispersive material having a second dielectric property;
wherein the second dielectric property differs from the first dielectric property to an extent sufficient to cause a discernible difference in a dielectric response to an applied electric field;
forming a first engineered microparticle using the first dielectrically-dispersive material; and
forming a second engineered microparticle using the second dielectrically- dispersive material.

34. The method of claim 33, wherein the first or second dielectrically-dispersive material comprises one or more layers of one or more materials.

35. The method of claim 33, wherein the first or second engineered microparticle comprises a dielectrically-dispersive core.

36. The method of claim 33, wherein the first or second engineered microparticle comprises a self-assembled monolayer.

37. The method of claim 33, wherein the first or second engineered microparticle comprises streptavidin.

38. The method of claim 37, wherein the first or second engineered microparticle comprises a biotinylated probe coupled to the streptavidin.

39. The method of claim 33, wherein the first or second engineered microparticle comprises a fluorescent label.

40. The method of claim 33, wherein the first or second engineered microparticle comprises a ganglioside.

41. The method of claim 33, wherein the first or second engineered microparticle comprises a vesicle.

42. The method of claim 41, wherein the vesicle comprises an erythrocyte ghost.

43. The method of claim 33, wherein modifying the first dielectrically-dispersive material comprises modifying an encapsulation material.

44. The method of claim 33, wherein modifying the first dielectrically-dispersive material comprises modifying a chain length.

45. The method of claim 33, wherein modifying the first dielectrically-dispersive material comprises modifying a doping agent.

46. The method of claim 33, wherein modifying the first dielectrically-dispersive material comprises modifying a method of manufacture.

47. The method of claim 46, wherein modifying the first dielectrically-dispersive material comprises modifying a heat treatments during manufacture.

48. The method of claim 33, wherein modifying the first dielectrically-dispersive material comprises modifying a composition.

49. The method of claim 33, wherein modifying the first dielectrically-dispersive material comprises modifying a surface charge.

50. The method of claim 33, wherein modifying the first dielectrically-dispersive material comprises modifying a side chain.

51. The method of claim 33, wherein modifying the first dielectrically-dispersive material comprises modifying a mobility of charge carriers.

52. The method of claim 33, wherein modifying the first dielectrically-dispersive material comprises modifying a viscosity.

53. An engineered microparticle comprising one or more gangliosides to affect microparticle aggregation.

54. The microparticle of claim 53, wherein the one or more gangliosides comprises a GM1 ganglioside.

55. The microparticle of claim 53, wherein the one or more gangliosides comprises a GD1a ganglioside

56. A method for controlling the aggregation of microparticles, comprising modulating the surface charge of one or more of the microparticles.

57. The method of claim 56, wherein modulating the surface charge comprises the addition of one or more gangliosides to the one or more microparticles.

58. The method of claim 57, wherein one or more of the gangliosides comprises a GM1 ganglio side.

59. The method of claim 57, wherein one or more of the gangliosides comprises a GD1a ganglioaside.

60. A method for identifying one or more complexes within a sample, the method comprising:
admixing with the sample a plurality of engineered microparticles, each microparticle comprising streptavidin and having a different dielectric property;
associating the plurality of engineered microparticles with one or more target analytes comprising biotin to form one or more complexes; and
identifying the one or more complexes by distinguishing between the different dielectric properties.
